(19) United States
(12) Patent Application Publication
(10) Pub. No.: US 2021/0023354 A1
(43) Pub. Date: Jan. 28, 2021

(54) METHODS AND APPARATUS FOR MICRONEEDLE INSERTION INTO TISSUE

(71) Applicant: Microdermics Inc., Vancouver (CA)

(72) Inventors: Iman MANSOOR, West Vancouver (CA); Sahan Anupama RANAMUKHAARACHCHI, Vancouver (CA); Boris STOEBER, Vancouver (CA); Mehrsa RAEISZADEH, North Vancouver (CA)

(21) Appl. No.: 16/845,053

(22) Filed: Apr. 9, 2020

Related U.S. Application Data
(63) Continuation of application No. PCT/CA2018/051307, filed on Oct. 17, 2018.
(60) Provisional application No. 62/573,570, filed on Oct. 17, 2017.

(51) Int. Cl. A61M 37/00

(52) U.S. Cl. CPC A61M 37/0015; A61M 2037/0061; A61M 2037/0023

(57) ABSTRACT

An apparatus for inserting a microneedle into tissue. The apparatus comprises microneedle supported by a backing to move therewith and a forcer that is selectably operable between a retracted state and an extended state. Upon operation of the forcer from the retracted state to the extended state, the forcer applies force to the backing which causes the backing to travel in an insertion direction toward the tissue. A releasable locking mechanism which, in a locking state, permits one-way motion of the backing in the insertion direction toward the tissue and lockingly engages the backing to prevent motion of the backing in a reverse direction opposed to the insertion direction, the releasable locking mechanism releasable, to a released state, which permits motion of the backing in the reverse direction. The forcer is disengaged from the backing when the locking mechanism lockingly engages the backing

### RELATED APPLICATIONS

This application is a continuation of Patent Cooperation Treaty (PCT) application No. PCT/CA2018/051307 having an international filing date of 17 Oct. 2018, which in turn claims priority from US application No. 62/573570 filed 17 Oct. 2017. Both PCT application No. PCT/CA2018/051307 and US application No. 62/573570 are hereby incorporated herein by reference.

### TECHNICAL FIELD

This invention relates to an apparatus and methods for fluid injection into tissue. Particular embodiments provide apparatus and methods for fluid injection into tissue by inserting one or more microneedles into tissue.

### BACKGROUND

Microneedles may be employed to deliver treatment such as fluid (e.g. drugs), electrical signals, or the like into tissue. Microneedles may also be employed for sensing particular compounds (e.g. biological fluid assays, drug concentration sensors, etc.) or extracting matter from tissue.

Various methods and apparatus have been disclosed for inserting microneedles into tissue. For example, see:
PCT application No PCT/IB2013/053708 published Nov. 14, 2013;
US Patent U.S. Pat. No. 7,252,651 B2 published Aug. 7, 2007;
EU patent EP2201969B1 published May 30, 2011;
PCT application No PCT/JP2015/085707 published Aug. 18, 2016;
US Patent US20140343502 published Nov. 20, 2014; and
US Patent US20080269666, published Oct. 30, 2008.

With some microneedle-insertion methods and apparatus, the microneedle is prone to “bounce-back” off of (i.e. away from) the tissue due to the elasticity of the tissue after insertion. In some cases, such “bounce-back” can result in ejection of the microneedle from the tissue in a direction generally opposing the insertion direction. Attempts have been made to counter such bounce-back by applying continuous force on the microneedle after insertion (i.e. in the insertion direction). However, when the microneedle is used for fluid injection, a wheal typically forms at or near the surface of the skin (e.g. to accommodate the volume of injected fluid). Application of continuous pressure in the insertion direction may prevent the tissue from expanding (e.g. may prevent a wheal from forming) and may therefore reduce effectiveness of the microneedle. For example, the rate of fluid injection may be reduced when pressure is applied against the formation of a wheal. In other situations (e.g. where microneedles are used for sensing or material extraction applications), application of force by the microneedle and/or parts of its insertion apparatus to the tissue may change one or more characteristics of the tissue and it may be desirable to reduce the force applied to the tissue after insertion of the microneedle to prevent or reduce such change to the one or more characteristics of the tissue. It may be generally desirable to reduce the force that is applied to the tissue by the microneedle and/or parts of its insertion apparatus after insertion of the microneedle, whether the microneedle is used for injection of fluid, a sensing application, a material extraction application and/or otherwise.

There is a general desire for simple, effective methods and apparatus for effective controlled insertion of needles and microneedles into tissue that reduce bounce-back of the microneedle upon insertion and , once inserted, minimize or reduce force that would tend to, undesirably affect characteristics of the tissue and/or counteract expansion of the tissue (e.g. wheal formation) upon the injection of fluid into the tissue.

The foregoing examples of the related art and limitations related thereto are intended to be illustrative and not exclusive. Other limitations of the related art will become apparent to those of skill in the art upon a reading of the specification and a study of the drawings.

### SUMMARY

One aspect of the invention provides an apparatus for inserting a microneedle into tissue. The apparatus may comprise a microneedle supported by a backing to move therewith, a forcer selectably operable between a retracted state and an extended state and a releasable locking mechanism. Upon operation of the forcer from the retracted state to the extended state, the forcer applies force to the backing which causes the backing to travel in an insertion direction toward the tissue. The releasable locking mechanism, in a locking state, permits one-way motion of the backing in the insertion direction toward the tissue and lockingly engages the backing to prevent motion of the backing in a reverse direction opposed to the insertion direction. The releasable locking mechanism is releasable, to a released state, which permits motion of the backing in the reverse direction. The forcer is disengaged from the backing when the locking mechanism lockingly engages the backing.

Another aspect of the invention provides an apparatus for inserting a microneedle into tissue. The apparatus comprises a housing for housing a microneedle, a backing for supporting a microneedle and moveable therewith relative to the housing, a forcer selectably operable between a retracted state and an extended state, and a forcer withdrawal mechanism. Upon operation of the forcer from the retracted state to the extended state, the forcer applies force to the backing which causes the backing to travel in an insertion direction toward the tissue and to thereby insert the microneedle into the tissue. At least a portion of the forcer withdrawal mechanism is couplable to the forcer after the microneedle is inserted into the tissue to move with the forcer and independently of the backing relative to the housing, to thereby disengage the forcer from the backing by movement of the portion of the forcer withdrawal mechanism and the forcer away from the backing in a reverse direction opposed to the insertion direction.

Another aspect of the invention provides an apparatus for inserting a microneedle into tissue. The apparatus comprises a microneedle supported by a backing to move therewith, a forcer selectably operable between a retracted state and an extended state, and an activator. Upon operation of the forcer from the retracted state to the extended state, the forcer applies force to the backing which causes the backing to travel in an insertion direction toward the tissue. When the activator is actuated, the activator enables the forcer to operate from the retracted state to the extended state wherein actuation of the activator comprises applying force to the activator in a transverse direction having at least a component orthogonal to the insertion direction.

Another aspect of the invention comprises an apparatus for inserting a microneedle into tissue. The apparatus comprises a housing for housing a microneedle, a backing for supporting a microneedle and moveable therewith relative to the housing, a forcer selectably operable between a retracted state and an extended state and wherein, upon operation of the forcer from the retracted state to the extended state, a proximal end of the forcer applies force to the backing which causes the backing to travel in an insertion direction toward the tissue and to thereby insert the microneedle into the tissue, and a forcer release mechanism, the forcer release mechanism releasable to allow movement of a distal end of the forcer, opposite to the proximal end, relative to the housing in a reverse direction opposed to the insertion direction to thereby reduce the force applied to the backing by the proximal end of the forcer to permit motion of the backing in the reverse direction.

In some embodiments, the locking mechanism comprises a lock that, in the locking state of the releasable locking mechanism, permits motion of at least a portion of the backing in the insertion direction from a location on a first side of the lock to a location on a second side of the lock and wherein interaction between the lock and the backing prevents motion of the at least a portion of the backing in the reverse direction from the location on the second side of the lock to the location on the first side of the lock. In some embodiments, the interaction comprises physical contact between the lock and the backing

In some embodiments, the locking mechanism is spaced apart from the backing when the forcer is in the retracted state and, wherein, the locking mechanism lockingly engages the backing as the backing travels in the insertion direction in response to the forcer operating from the retracted state to the extended state

In some embodiments, the backing comprises at least one concavity and the locking mechanism comprises at least one pawl which extends into the at least one concavity to lockingly engage the backing as the backing travels in the insertion direction in response to the forcer operating from the retracted state to the extended state.

In some embodiments, the backing comprises a plurality of transversely extending teeth, each transversely extending tooth comprising an insertion-direction face and a reverse-direction face and wherein each concavity is defined by the insertion-direction face and the reverse-direction face of a pair of adjacent teeth.

In some embodiments, the insertion-direction face of each tooth is shaped such that contact between the pawl and the insertion-direction face of each tooth causes movement (e.g. translation and/or rotation) of at least part of the pawl as the backing travels in the insertion direction in response to the forcer operating from the retracted state to the extended state.

In some embodiments, the insertion-direction face of each tooth is shaped such that contact between the pawl and the insertion-direction face of each tooth causes deformation of a bias mechanism that biases the pawl toward the backing as the backing travels in the insertion direction in response to the forcer operating from the retracted state to the extended state.

In some embodiments, the reverse-direction face of each tooth is shaped to guide the pawl into a corresponding concavity and thereby prevent reverse direction movement of the backing.

In some embodiments, the pawl is shaped to extend transversely and in the insertion direction when the locking mechanism lockingly engages the backing.

In some embodiments, at least a portion of the pawl is biased in a transverse direction toward the backing when the locking mechanism lockingly engages the backing.

In some embodiments, the pawl is pivotally mounted and wherein the portion of the pawl is pivotally biased toward the backing when the locking mechanism lockingly engages the backing.

In some embodiments, at least a portion of the pawl is deformed in a transverse direction away from the backing when the locking mechanism lockingly engages the backing

In some embodiment, the apparatus comprises a pawl release which is actuatable to withdraw the pawl from engagement with the concavity.

In some embodiments, the pawl release is actuatable to withdraw the pawl from engagement with the concavity by pivotal motion of the pawl.

In some embodiments, the locking mechanism is releasable by applying force to the housing in a transverse direction nonparallel to the insertion and reverse directions.

In some embodiments, the locking mechanism is releasable by applying force to the housing in the insertion direction.

In some embodiments, the housing comprises a first section having a first beveled surface and a second section having a second beveled surface and application of force to the housing in the insertion direction causes the first beveled surface of the first section of the housing to contact the second beveled surface of the second section of the housing thereby deforming and/or otherwise moving at least a portion of the second section of the housing to release the locking mechanism.

In some embodiments, deforming and/or otherwise moving at least a portion of the second section of the housing to release the locking mechanism comprises deforming and/or otherwise moving the at least a portion of the second section of the housing in a transverse direction nonparallel to the insertion and reverse directions.

In some embodiments, deforming and/or otherwise moving at least a portion of the second section of the housing to release the locking mechanism release comprises deforming and/or otherwise moving the at least a portion of the second section of the housing until one or more stops projecting from the second section of the housing disengage the backing.

In some embodiments, deforming and/or otherwise moving at least a portion of the second section of the housing to release the locking mechanism comprises deforming and/or otherwise moving the at least a portion of the second section of the housing until one or more stops projecting from the backing disengage the second section of the housing.

In some embodiments, deforming and/or otherwise moving at least a portion of the second section of the housing to release the locking mechanism comprises deforming and/or otherwise moving the at least a portion of the second section of the housing until one or more teeth projecting from the second section of the housing disengage the backing.

In some embodiments, the forcer applies force to the backing which causes the backing to travel in the insertion direction toward the tissue as a projectile which is disengaged from the forcer during at least a portion of the travel of the backing in the insertion direction.

In some embodiments, the apparatus comprises a catch which prevents the forcer from operating from the retracted state to the extended state.

In some embodiments, the catch is releasable to allow the forcer to operate from the retracted state to the extended state by applying force to the housing in a transverse direction nonparallel to the insertion and reverse directions.

In some embodiments, the catch is releasable to allow the forcer to operate from the retracted state to the extended state by applying force to the housing in the insertion direction.

In some embodiments, applying force to the housing in the insertion direction comprises forcing a third beveled surface of a first section of the housing to contact a fourth beveled surface of a second section of the housing thereby deforming and/or otherwise moving at least a portion of the second section of the housing to release the catch.

In some embodiments, deforming and/or otherwise moving at least a portion of the second section of the housing to release the catch comprises deforming and/or otherwise moving the at least a portion of the second section of the housing in a radially outward direction nonparallel to the insertion and reverse directions.

In some embodiments, deforming and/or otherwise moving at least a portion of the second section of the housing to release the catch comprises deforming and/or otherwise moving the at least a portion of the second section of the housing until the catch projecting from the second section of the housing disengages the backing thereby enabling the forcer to operate from the retracted state to the extended state.

In some embodiments, deforming and/or otherwise moving at least a portion of the second section of the housing to release the catch comprises deforming and/or otherwise moving the at least a portion of the second section of the housing until the catch projecting from backing disengages the second section of the housing thereby enabling the forcer to operate from the retracted state to the extended state.

In some embodiments, the apparatus comprises a fluid conduit connected to transport fluid to or from the microneedle when the forcer is in the retracted state and when the forcer is in the extended state.

In some embodiments, the forcer withdrawal mechanism is free to move relative to the forcer when the forcer is in the retracted state.

In some embodiments, the forcer is located within the housing.

In some embodiments, the apparatus comprises a catch which prevents the forcer from operating from the retracted state to the extended state.

In some embodiments, the catch is releasable to allow the forcer to operate from the retracted state to the extended state by applying force to the housing in a transverse direction nonparallel to the insertion and reverse directions.

In some embodiments, the catch is releasable to allow the forcer to operate from the retracted state to the extended state by applying force to the housing in the insertion direction.

In some embodiments, the portion of the forcer withdrawal mechanism is coupleable to the forcer by abutting the portion of the forcer withdrawal mechanism against the forcer.

In some embodiments, the forcer comprises a store of potential energy and wherein at least some of the potential energy remains in the store after insertion of the microneedle into the tissue.

In some embodiments, the apparatus comprises a forcer locking mechanism for lockingly engaging the portion of the forcer withdrawal mechanism in a location where the forcer is spaced apart from the backing and for preventing movement of the forcer in the insertion direction.

In some embodiments, a force applied to the backing by the forcer in the insertion direction after the microneedle is inserted into the tissue is greater than any bounce-back force associated with restoring deformation of the tissue that may have occurred as a result of insertion of the microneedle into the tissue.

In some embodiments, applying force to the activator in a transverse direction comprises applying force to a housing in the transverse direction.

In some embodiments, the backing comprises an adhesive surface contactable with the tissue when the microneedle is inserted into the tissue.

In some embodiments, the apparatus comprises an activator which, when actuated, enables the forcer to operate from the retracted state to the extended state.

In some embodiments, actuation of the activator comprises applying force to the housing in the insertion direction.

In some embodiments, releasing the forcer release mechanism comprises applying force to the housing in the insertion direction.

In some embodiments, a force applied to the backing by the forcer in the insertion direction after the microneedle is inserted into the tissue is greater than any bounce-back force associated with restoring deformation of the tissue that may have occurred as a result of insertion of the microneedle into the tissue. In some embodiments, the catch is releasable to allow the forcer to operate from the retracted state to the extended state by applying force to the housing in a transverse direction nonparallel to the insertion and reverse directions. In some embodiments, the catch is releasable to allow the forcer to operate from the retracted state to the extended state by applying force to the housing in the insertion direction. In some embodiments, applying force to the housing in the insertion direction comprises forcing a third beveled surface of a first section of the housing to contact a fourth beveled surface of a second section of the housing thereby deforming and/or otherwise moving at least a portion of the second section of the housing to release the catch. In some embodiments, deforming and/or otherwise moving at least a portion of the second section of the housing to release the catch comprises deforming and/or otherwise moving the at least a portion of the second section of the housing in a radially outward direction nonparallel to the insertion and reverse directions. In some embodiments, deforming and/or otherwise moving at least a portion of the second section of the housing to release the catch comprises deforming and/or otherwise moving the at least a portion of the second section of the housing until the catch projecting from the second section of the housing disengages the backing thereby enabling the forcer to operate from the retracted state to the extended state. In some embodiments, deforming and/or otherwise moving at least a portion of the second section of the housing to release the catch comprises deforming and/or otherwise moving the at least a portion of the second section of the housing until the catch projecting from backing disengages the second section of the housing thereby enabling the forcer to operate from the retracted state to the extended state.

Another aspect of the invention provides a method for inserting a microneedle into tissue. The method comprises supporting a microneedle by a backing to move therewith, selectably operating a forcer between a retracted state and an extended state, lockingly engaging the backing with a releasable locking mechanism and disengaging the forcer from the backing. Force is applied to the backing via the forcer by selectably operating the forcer from the retracted state to the extended state to thereby cause the backing to travel in an insertion direction toward the tissue. The releasable locking mechanism lockingly engages the backing to, in a locking state, prevent motion of the backing in a reverse direction opposed to the insertion direction while still permitting one-way motion of the backing in the insertion direction toward the tissue, the releasable locking mechanism releasable, to a released state, which permits motion of the backing in the reverse direction.

In some embodiments, selectably operating the forcer comprises applying a first force to the housing in a first transverse direction having at least a component orthogonal to the insertion direction.

In some embodiments, releasing the locking mechanism from the backing.

In some embodiments, releasing the locking mechanism from the backing by withdrawing the first force.

In some embodiments, lockingly engaging the backing comprises applying a second force to the locking mechanism in a second transverse direction having at least a component orthogonal to the insertion direction.

In some embodiments, applying the second force to the locking mechanism comprises applying the first force to the housing.

Another aspect of the invention provides a method for inserting a microneedle into tissue. The method comprises housing a microneedle in a housing, supporting a microneedle by a backing to move therewith, selectably operating a forcer between a retracted state and an extended state, applying force to the backing via the forcer by operating the forcer from the retracted state to the extended state to cause the backing to travel in an insertion direction toward the tissue to thereby insert the microneedle into the tissue, withdrawing the forcer using a forcer withdrawal mechanism. Withdrawing the forcer using the forcer withdrawal mechanism comprises coupling at least a portion of the forcer withdrawal mechanism to the forcer after the microneedle is inserted into the tissue and moving the forcer by moving of the portion of the forcer withdrawal mechanism and the forcer away from the backing and relative to the housing in a reverse direction opposed to the insertion direction to thereby disengage the forcer from the backing.

In some embodiments, selectably operating the forcer comprises applying a first force to the housing in a first transverse direction having at least a component orthogonal to the insertion direction.

In some embodiments, after moving the forcer by moving the portion of the forcer withdrawal mechanism, locking the forcer withdrawal mechanism relative to the housing to prevent the forcer from applying force to the backing.

In some embodiments, locking the forcer withdrawal mechanism comprises twisting the forcer withdrawal mechanism relative to the housing.

Another aspect of the invention provides a method for inserting a microneedle into tissue. The method comprises supporting a microneedle by a backing to move therewith, selectably operating a forcer between a retracted state and an extended state, applying force to the backing via the forcer by operating the forcer from the retracted state to the extended state to thereby cause the backing to travel in an insertion direction toward the tissue, and actuating an actuator to enable the forcer to operate from the retracted state to the extended state.

In some embodiments, actuating the activator comprises applying force to the activator in a transverse direction having at least a component orthogonal to the insertion direction.

In some embodiments, the method comprises contacting an adhesive surface of the backing to the tissue to adhere the backing to the tissue.

Another aspect of the invention comprises a method for inserting a microneedle into tissue. The method comprises housing a microneedle in a housing, supporting a microneedle by a backing to move therewith, selectably operating a forcer between a retracted state and an extended state, applying force to the backing via the forcer by operating the forcer from the retracted state to the extended state to cause the backing to travel in an insertion direction toward the tissue to thereby insert the microneedle into the tissue, and releasing a distal end of the forcer, opposite to the proximal end, to allow movement of the distal end of the forcer relative to the housing in a reverse direction opposed to the insertion direction thereby reducing the force applied to the backing by the proximal end of the forcer to permit motion of the backing in the reverse direction.

Other aspects of the invention are provided in the detailed description that follows.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIGS. 1 A to 1 E (collectively FIG. 1) depict schematic illustrations of an apparatus for inserting a microneedle into tissue according to one embodiment of the invention.

FIGS. 2A to 2C (collectively FIG. 2) depict a locking mechanism for an apparatus for inserting a microneedle into tissue according to one embodiment of the invention.

FIGS. 3A to 3D (collectively FIG. 3) depict schematic illustrations of an apparatus for inserting a microneedle into tissue according to one embodiment of the invention.

FIGS. 4A to 4D (collectively FIG. 4) depict schematic illustrations of an apparatus for inserting a microneedle into tissue according to one embodiment of the invention.

FIG. 5A depicts an elevated view of an apparatus for inserting a microneedle into tissue according to one embodiment of the invention.

FIGS. 5B to 5F depict cross-sectional views of the apparatus of FIG. 5A.

FIGS. 6A to 6C (collectively FIG. 6) depict schematic illustrations of an apparatus for inserting a microneedle into tissue according to one embodiment of the invention.

FIGS. 7A to 7E (collectively FIG. 7) depict schematic illustrations of an apparatus for inserting a microneedle into tissue according to one embodiment of the invention.

FIGS. 8A to 8E (collectively FIG. 8) depict schematic illustrations of an apparatus for inserting a microneedle into tissue according to the FIG. 7 embodiment of the invention.

FIGS. 9A to 9D (collectively FIG. 9) depict schematic illustrations of a release mechanism according to one embodiment of the invention.

FIGS. 10A to 10D (collectively FIG. 10) depict schematic illustrations of an apparatus for inserting a microneedle into tissue according to one embodiment of the invention.

FIGS. 11A to 11 D (collectively FIG. 11) depict schematic illustrations of an apparatus for inserting a microneedle into tissue according to the FIG. 9 embodiment of the invention.

### Description

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Particular aspects of the invention provide methods and apparatus for microneedle insertion into tissue. In particular embodiments, an apparatus is provided comprising a microneedle supported by a backing such that the microneedle may move with the backing. A forcer is selectively operable between a retracted state and an extended state. In its extended state, the forcer causes the backing to travel in a first direction toward the tissue. A locking mechanism is provided which permits one-way motion of the backing in a first direction toward the tissue. The locking mechanism lockingly engages the backing to prevent motion of the backing in a reverse direction away from the tissue.

In another particular embodiment, a housing is provided for housing a microneedle. A backing is provided for supporting the microneedle. The backing is moveable with the microneedle relative to the housing. A forcer is selectively operable between a retracted state and an extended state. In its extended state, the forcer causes the backing to travel in a first direction toward the tissue. A forcer withdrawal mechanism is provided and is free to move relative to the forcer prior to insertion of the microneedle. The forcer withdrawal mechanism is couplable to the forcer after the microneedle is inserted. When coupled to the forcer, the forcer withdrawal mechanism moves with the forcer relative to the housing to thereby disengage the forcer from the backing by movement of the forcer away from the tissue.

In another particular embodiment, an apparatus is provided comprising a microneedle supported by a backing such that the microneedle may move with the backing. A forcer is selectively operable between a retracted state and an extended state. In its extended state, the forcer causes the backing to travel in a first direction toward the tissue. An adhesive is provided on a surface of the backing such that upon contact with a tissue surface, the adhesive adheres the backing to the tissue surface to prevent motion of the backing in a reverse direction away from the tissue.

In some embodiments, the forcer switches from the retracted state to the extended state when a force is applied to the housing or an activator in a third direction that is non-parallel to the first direction.

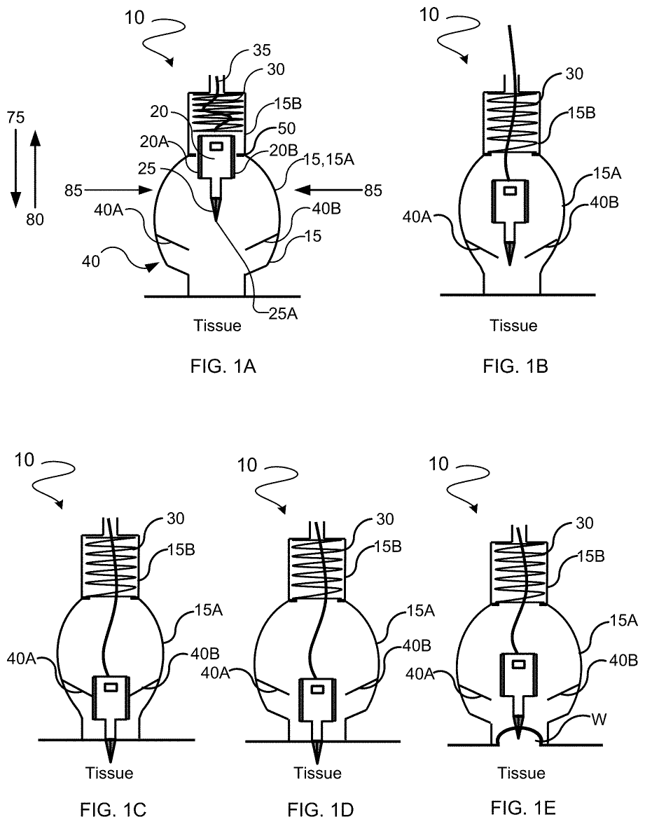

FIG. 1A depicts an apparatus 10 for inserting a microneedle 25 into tissue according to a particular non-limiting embodiment of the invention. In the illustrated embodiment, microneedle 25 is supported by a backing 20. A forcer 30 is selectably operable between a retracted state (as depicted in FIG. 1A) and an extended state (as depicted in FIG. 1 B). When forcer 30 operates from its retracted state to its extended state, forcer 30 applies force against backing 20 which causes backing 20 to travel in direction 75 toward the tissue and to insert microneedle 25 into the tissue of an animal (e.g. a human). The direction 75 shown by the corresponding arrow in FIG. 1A may be referred to as the insertion direction, since this is the direction that microneedle 25 is inserted into the tissue. Microneedle injection apparatus 10 shown in the FIG. 1 embodiment comprises a locking mechanism 40 which permits one-way motion of backing 20 in the injection direction 75 and lockingly engages backing 20 to prevent motion of backing 20 in a reverse direction 80 opposed to insertion direction 75.

Microneedle 25 may comprise any suitable needle known in the art. In currently preferred embodiments, microneedle 25 is a microneedle having a length of less than 3 mm or less than 2 mm and a cross-section of less than 250 μm at its distal (away from its point) end. This is not mandatory. Microneedle 25 may be solid or hollow. Microneedle 25 may be metallic. Microneedle 25 may be configured to deliver fluid to the tissue via a conduit 35 which may be in fluid communication with a fluid reservoir. In some embodiments, conduit 35 comprises a piston or other suitable mechanism for forcing fluid through conduit 35 and out the tip 25A of microneedle 25. In some embodiments, conduit 35 is unnecessary and microneedle 25 is connected directly to a fluid reservoir that may or may not move with microneedle 25.

In the FIG. 1 embodiment, backing 20, microneedle 25 and/or at least some of forcer 30 may be contained in a housing 15. In some embodiments, housing 15 comprises an elastically deformable material such as a polymeric material, a composite material, silicon, rubber, etc. In some embodiments, housing 15 comprises metal, glass or another non-polymeric material. In some embodiments, housing 15 is substantially closed prior to use of apparatus 10 to prevent contamination of microneedle 25 and/or other internal components of apparatus 10, such as conduit 35. In some embodiments, housing 15 may have one or more openings for accessing internal parts of apparatus 10 (such as microneedle 25 or locking mechanism 40). In some embodiments, housing 15 is integrated into a multi-purpose medical device. Housing 15 may be dimensioned so that housing 15 itself does not interfere with expansion of the tissue (e.g. formation of a wheal) or contraction of the tissue (e.g. due to extraction of matter from the tissue).

Forcer 30 may comprise any suitable forcer capable of applying force against backing 20 in insertion direction 75. For example, forcer 30 may comprise a spring, as depicted in FIGS. 1A-1D. In some embodiments, forcer 30 may additionally or alternatively comprise apparatus for application of force against backing 20 using other techniques such as compressed gas, gravity, or electricity. In some embodiments, forcer 30 may additionally or alternatively comprise one or more magnets, one or more electromagnets, a solenoid actuator, a voice coil actuator, an elastomeric material, a manually operated piston, a pneumatic actuator, a magnetic actuator, a hydraulic actuator, etc. Forcer 30 may comprise a store of potential energy wherein at least a portion of the potential energy stored in forcer 30 is released as forcer 30 operates from its retracted configuration to its extended configuration. In some embodiments, forcer 30 is fully extended in the extended configuration while in other embodiments, the extended configuration of forcer 30 may comprise only a partial extension of forcer 30 and forcer 30 may be capable of extending further. Such extension of forcer 30 may be limited by other factors such as, for example, if forcer 30 or backing 20 contacts something which prevents further extension of forcer 30.

Various methods and mechanisms may be employed to change the state of forcer 30 from the retracted state to the expanded state. In some embodiments, the state of forcer 30 may be changed by activating forcer 30. For example, an electrical current may be supplied to a solenoid actuator causing the solenoid actuator to apply force to backing 20 such that backing 20 travels in insertion direction 75 (e.g. toward the tissue).

In some embodiments, forcer 30 is naturally biased toward its extended state and is held in the retracted state by one or more stops or stop-mechanisms (also referred to herein as a catch). For example, in the FIG. 1 embodiment, forcer 30 may be naturally biased toward its extended state (e.g. the spring of forcer 30 may be compressed) but forced 30 may be held in its retracted state by one or more stops 50. In the FIG. 1 embodiment, stops 50 may physically engage (e.g. abut) backing 20 to prevent backing 20 from moving in insertion direction 75 or to otherwise prevent forcer 30 from applying force which would tend to move backing 20 in insertion direction 75. When stops 50 engage backing 20 (as is the case in FIG. 1A), backing 20 in turn prevents forcer 30 from activating or extending. By releasing stops 50, forcer 30 may be free to activate or expand and may apply force which tends to move backing 20 in insertion direction 75. In some embodiments, stops 50 may additionally or alternatively engage forcer 30 directly to prevent backing 20 from moving in insertion direction 75 or to otherwise prevent forcer 30 from applying force which would tend to move backing 20 in insertion direction 75.

Stops 50 may comprise any suitable stopping mechanism. In some embodiments, stops 50 comprise one or more protrusions or flanges extending from an interior surface of housing 15 (or another part of apparatus 10). Stops 50 may physically engage (e.g. abut against) backing 20 or forcer 30. Stops 50 may be physically disengaged from backing 20 or forcer 30 using any suitable technique. In the illustrated embodiment of FIG. 1A, stops 50 may be disengaged from backing 20 or forcer 30 by deforming (either elastically or plastically) at least a portion of housing 15. In some embodiments, stops 50 may comprise one or more moveable parts releasable by pivoting, sliding, ratcheting, twisting, compressing or the like. Stops 50 may comprise one or more solenoid actuators or the like. In some embodiments, stops 50 may physically engage a flange (not shown) of backing 20 and/or sides 20A, 20B of backing 20. In some embodiments, stops 50 engage one or more teeth on sides 20A, 20B.

In the FIG. 1 embodiment, stops 50 are released from backing 20 by applying force to housing 15 in one or more transverse directions 85 non-parallel to (e.g. having components orthogonal to) insertion direction 75. In some embodiments, transverse directions 85 are orthogonal to insertion direction 75. In some embodiments, transverse directions 85 have components that are orthogonal to insertion direction 75. In some embodiments, force is applied to squeeze or pinch portion 15A of housing 15 to deform portion 15A. As portion 15A deforms, portion 15B of housing 15 may also deform, thereby relieving the engagement of (disengaging) stops 50 from sides 20A, 20B of backing 20 When stops 50 disengage from sides 20A, 20 of backing 20, forcer 30 applies force which tends to move backing 20 in insertion direction 75 past stops 50. In some embodiments, deformation of portions 15A, 15B is elastic and restorative deformation of portions 15A, 15B (e.g. deformation returning portions 15A, 15B to their non-deformed shapes or close thereto) occurs after the force on portion 15A is relieved. This is not mandatory.

Stops 50 may also serve to limit extension of forcer 30 in insertion direction 75. For example, as can be seen from FIG. 1 B, as forcer 30 extends, forcer 30 is prevented from moving past stops 50 in insertion direction 75. While the extension of forcer 30 in insertion direction 75 is stopped, backing 20 may disengage from forcer 30 and continue to travel in insertion direction 75. In this manner, forcer 30 of the illustrated embodiment “fires” backing 20 (e.g. as a projectile) in insertion direction 75. In some embodiments, the functions of stops 50 (i.e. engaging backing 20 and/or forcer 30 prior to extension of forcer 30 and limiting the extent of extension of forcer 30) are implemented by two different mechanisms. In some embodiments, stops 50 handle both of these functions.

Once stops 50 have released backing 20 and/or forcer 30 has been activated, forcer 30 applies force against backing 20 in insertion direction 75, thereby causing backing 20 (and microneedle 25) to move in insertion direction 75 toward the tissue. In the FIG. 1 embodiment, as forcer 30 contacts stops 50 and the extent of its extension is limited (or as the extent of extension of forcer 30 is otherwise limited), backing 20 disengages from forcer 30 and continues to travel (e.g. as a projectile) in insertion direction 75 toward the tissue.

In the FIG. 1 embodiments, where forcer 30 is implemented using a spring, the magnitude of the force applied by forcer 30 to backing 20 and the corresponding velocity of backing 20 (when it is released as a projectile) may be dependent on the spring constant associated with the spring of forcer 30 and the linear compression of the spring of forcer 30 (between the retracted configuration of FIG. 1A and the extended configuration of FIG. 1 B). This compression distance may be described as the displacement distance between the position of backing 20 when forcer 30 is in the retracted state (FIG. 1A) and the position of backing 20, when backing 20 is released (fired as a projectile) from forcer 30. In some embodiments, backing 20 is released (fired as a projectile) before forcer 30 is fully extended. In other embodiments, forcer 30 is fully extended when backing 20 is released (fired as a projectile). In embodiments like the FIG. 1 embodiment (where backing 20 and microneedle 25 are fired toward the tissue as projectiles), these factors which influence the motion (e.g. velocity) of backing 20 and microneedle 25 when they are released as a projectile may be referred to as the projection parameters of forcer 30. It will be appreciated by those skilled in the art, that other types of forcers 30 may have other types of projection parameters.

Gravitational forces and frictional forces may also impact the motion of backing 20 and microneedle 25. Once microneedle 25 and/or backing 20 contacts the tissue, the tissue will also exert force on microneedle 25 and/or backing 20. Such forces exerted by the tissue may include a friction-like resistance to insertion of microneedle 25 into the tissue and may also include a “bounce-back” force associated with restoring any deformation of the tissue caused by the impact of microneedle 25 and/or backing 20. Such forces exerted by the tissue tend to be oriented in reverse direction 80, opposed from insertion direction 75. In some embodiments, it may be desirable to adjust the projection parameters of forcer 30 to ensure sufficient insertion of microneedle 25 and/or to reduce bounce-back.

As backing 20 travels in insertion direction 75 toward the tissue, locking mechanism 40 may engage backing 20 to mitigate against bounce-back. In the illustrated embodiment of FIG. 1, as backing 20 travels in insertion direction 75 toward the tissue, backing 20 may pass from an unlocked configuration where backing 20 is not engaged by locking mechanism 40 (as depicted in FIG. 1A) to a locked configuration where backing 20 is engaged by locking mechanism 40 (as depicted in FIG. 1B). As can be seen from FIGS. 1A and 1B, backing 20 and locking mechanism 40 are in the unlocked configuration when backing 20 is relatively spaced apart from the tissue (in insertion direction 75) and are in the locked configuration when backing is relatively close (in insertion direction 75) to the tissue. In the unlocked configuration, backing 20 is free to move without such motion being influenced by locking mechanism 40. In the locked configuration, locking mechanism 40 lockingly engages backing 20 to prevent motion of backing 20 in reverse direction 80 opposed to insertion direction 75, while still allowing motion of backing 20 in insertion direction 75 toward the tissue. Locking mechanism 40 may therefore reduce bounce-back while allowing for backing 20 (and microneedle 25) to travel at sufficient velocity to ensure penetration of the tissue by microneedle 25.

Locking mechanism 40 may comprise any suitable locking mechanism for effecting such one-way motion of backing 20 and microneedle 25. For example, locking mechanism 40 may comprise an electrically driven solenoid that activates when the presence of backing 20 is sensed by one or more sensors and permits motion of backing 20 in insertion direction 75 while preventing motion of backing 20 in reverse direction 80. In another example, locking mechanism 40 may comprise one or more magnets or electromagnets which permit motion of backing 20 in insertion direction 75 and prevent motion of backing 20 in reverse direction 80. In some embodiments like the FIG. 1 embodiment, locking mechanism 40 comprises at least one pawl lockingly engageable in at least one concavity. In the FIG. 1 embodiment, side 20A of backing 20 comprises at least one concavity engageable by a first pawl 40A of locking mechanism 40 and side 20B of backing 20 comprises at least one concavity engageable by a second pawl 40B of locking mechanism 40. In some embodiments, pawls 40A, 40B may be located to engage the corresponding sides 20A, 20B. In some embodiments, pawls 40A, 40B may be biased (e.g. by suitable biasing mechanisms) toward corresponding sides 20A, 20B.

The concavities on sides 20A, 20B of backing 20 may be formed by one or more teeth on sides 20A, 20B. To allow backing 20 to travel in insertion direction 75, surfaces of the one or more teeth that face at least partially in insertion direction 75 (e.g. have normal vectors with components in insertion direction 75) may be beveled such that the first and second pawls 40A, 40B slide relatively easily (e.g. without significant decelerating force in reverse direction 80) along the beveled portion of the one or more teeth as backing 20 travels in insertion direction 75. In some embodiments, such motion of backing 20 in insertion direction 75 may comprise deformation of one or both of pawls 40A, 40B and/or the deformation of the biasing mechanisms associated with one or both of pawls 40A, 40B.

In some embodiments, the bevel angle of the surface of pawls 40A, 40B may additionally or alternatively be shaped to permit motion of backing 20 relative to pawls 40A, 40B in insertion direction 75.

On the other hand, to prevent backing 20 from travelling in reverse direction 80 opposed to insertion direction 75, surfaces of the one or more teeth that face at least partially in reverse direction 80 (e.g. have normal vectors with components in reverse direction 80) may be beveled such that backing 20 is incapable of sliding relative to first and second pawls 40A, 40B to travel in reverse direction 80. In some embodiments, the bevel angle of the surface of pawls 40A, 40B may additionally or alternatively be shaped to prevent motion of backing 20 relative to pawls 40A, 40B in reverse direction 80.

In the locked configuration, locking mechanism 40 permits motion of at least a portion of backing 20 in insertion direction 75 from a location on a first side of pawls 40A, 40B to a location on a second side (opposing the first side) of pawls 40A, 40B and interaction between pawls 40A, 40B and backing 20 prevents motion of the at least a portion of backing 20 in reverse direction 80 from a location on the second side of pawls 40A, 40B to a location on the first side of pawls 40A, 40B.

In practice, as forcer 30 extends from its retracted configuration (FIG. 1A) to its extended configuration (FIG. 1 B), backing 20 is caused to move in insertion direction 75 toward the tissue by the application of force by forcer 30. In the illustrated FIG. 1 embodiment, forcer 30 encounters stops 50 which prevent further extension of forcer 30, thereby causing forcer 30 to “fire” backing 20 as a projectile. Backing 20 continues to move in insertion direction 75 until microneedle 25 is inserted into the tissue. Microneedle 25 is prevented from withdrawing from the tissue (in reverse direction 80) by locking engagement of locking mechanism 40 with backing 20. Accordingly, bounce-back of microneedle 25 out of the tissue is reduced as compared to other methods and apparatus.

After microneedle 25 is inserted into the tissue as desired, microneedle 25 may be used to, for example, inject fluid into the tissue, apply electrical current to the tissue, extract matter from the tissue, sensing and monitoring interstitial fluid (ISF), blood, skin composition etc. In the FIG. 1 embodiment, fluid may be injected into the tissue via conduit 35. As fluid is injected into the tissue, the tissue may begin to expand (e.g. the tissue may form a wheal, W). To allow the tissue to expand without interference from backing 20, locking mechanism 40 may be released before injection of fluid into the tissue.

Locking mechanism 40 may be released by any suitable mechanism or using any suitable method. In some embodiments, locking mechanism 40 may be released by withdrawing forces applied to housing 15 in directions 85, to thereby allow housing 15 to restoratively deform to its un-deformed shape or to a shape similar to its un-deformed state.

In some embodiments, with housing 15 in an un-deformed state, first and second pawls 40A, 40B of locking mechanism 40 are sufficiently close together to lockingly engage backing 20. In some embodiments, first and second pawls 40A, 40B are only close enough to one another to lockingly engage backing 20 upon deformation of housing 15. Such deformation may be caused by force applied to housing 15 in directions 85. In some embodiments, the force applied to portion 15A of housing 15 to release stops 50 from backing 20 may be sufficient to allow first and second pawls 40A, 40B to lockingly engage backing 20.

In such embodiments, a user may squeeze portion 15A of housing 15 (e.g. in transverse directions 85) to release stops 50 from backing 20 (causing forcer 30 to extend to its extended configuration and to move backing 20 in insertion direction 75) and may continue to squeeze portion 15A until after microneedle 25 is inserted into the tissue as desired and until any bounce-back forces have subsided. Then, by removing the force applied to portion 15A and allowing housing 15 to at least partially restoratively deform (i.e.

return to its un-deformed shape), pawls 40A, 40B may move apart from one another such that locking mechanism 40 is released from backing 20 and backing 20 is free to move in reverse direction 80 as the tissue expands, and/or as wheal, W, forms.

In some embodiments, to ensure that sufficient transverse direction 85 force is applied to housing 15 to engage locking mechanism 40, housing 15 may be constructed such that it may elastically deform upon application of force in directions 85 until a certain point wherein a rigid (or semi-rigid) stop prevents additional deformation. In this way, a user may discern that sufficient force has been applied to housing 15 to engage locking mechanism 40 when it is no longer possible (or becomes noticeably more difficult) to deform housing 15 in directions 85. Such a rigid (or semi-rigid) stop may be achieved in various ways, such as with an internal skeleton, a rigid internal stop, or through material choice. In some embodiments, the same amount of deformation of housing 15 may be used to release stops 50 and to engage locking mechanism 40.

Locking mechanism 40 may be released in other ways. For example, in the case that locking mechanism 40 comprises one or more solenoid actuators, the actuators may be caused to release by the push of a button or in response to information from one or more sensors.

Once locking mechanism 40 is released, backing 20 is permitted to move in reverse direction 80. Fluid may be injected into the tissue and the tissue may be allowed to expand in reverse direction 80 (e.g. form a wheal W), as depicted in FIG. 1E, without being impeded by backing 20 or locking mechanism 40.

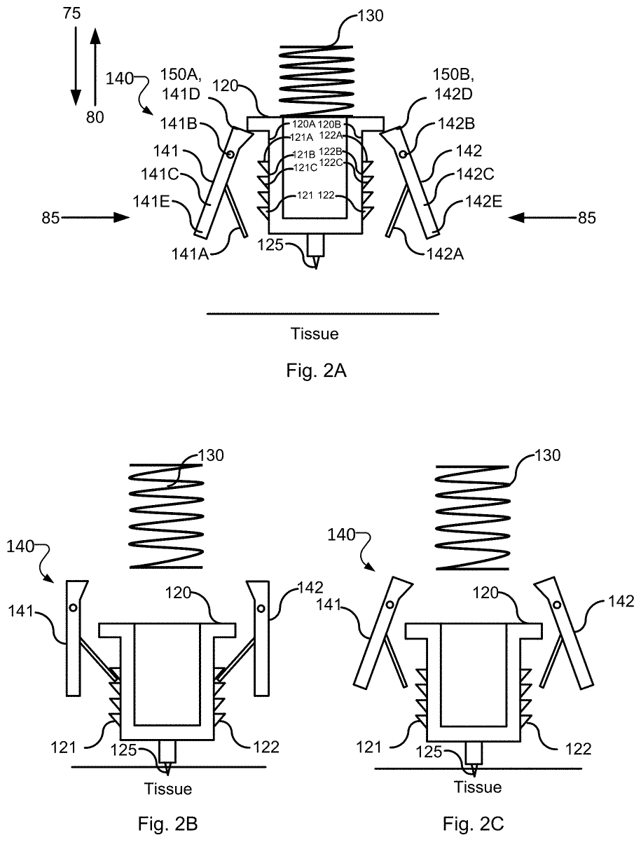

FIGS. 2A to 2C depict an exemplary locking mechanism 140 according to a particular embodiment of the invention. The FIG. 2 locking mechanism 140 may be used in the FIG. 1 apparatus 10. Locking mechanism 140 comprises a first lock 141 and a second lock 142. First and second locks 141, 142 are pivotably mounted at pivots 141B, 142B respectively. In some embodiments, first and second locks 141, 142 are pivotably mounted to a housing (e.g. housing 15 of the FIG. 1 apparatus 10) while in other embodiments, first and second locks 141, 142 are pivotably mounted to another portion of the apparatus.

A biasing member (e.g. a spring, torsion spring, elastomeric element, or the like) may apply force to first and second locking members 141, 142 (e.g. torque around pivots 141B, 142B), such that ends 141D, 142D of locking members 141, 142 are biased toward each other and ends 141E, 142E of locking members 141, 142 are biased apart from one another.

In some embodiments, ends 141D, 142D may function as first and second stops 150A, 150B (similar to stops 50). In such embodiments, first and second stops 150A, 150B are biased toward backing 120 such that a portion of backing 120 abuts each of first and second stops 150A, 150B thereby preventing movement of backing 120 in direction 75.

First and second locks 141, 142 comprise first and second pawls 141A, 142A. In some embodiments, first and second pawls 141A, 142A are provided by ends 141 E, 142E of first and second locks 141, 142. In other embodiments, first and second pawls 141A, 142A are provided by other projecting members, such as, for example, is depicted in the FIG. 2 embodiment. First and second pawls 141A, 142A may be somewhat flexible to allow for deformation of pawls 141A, 142A as teeth of backing 120 pass by first and second pawls 141A, 142A in insertion direction 75.

As ends 141D, 142D are biased toward each other (e.g. toward backing 120), first and second pawls 141A, 142A are biased apart from one another (e.g. away from backing 120). The biasing force at pivots 141B, 142B may be overcome by applying force in transverse directions 85 to portions 141C, 142C of locks 141, 142. Portions 141C, 142C are located on opposite sides of pivots 141B, 142B as compared to ends 141 D, 142D. As the biasing force at pivots 141 B, 142B is overcome, stops 150A, 150B move apart from one another thereby allowing backing 120 to move in insertion direction 75 due to the force applied by forcer 130. Meanwhile, as stops 150A, 150B move apart, pawls 141A, 142A move together such that pawls 141A, 142A engage sides 120A, 120B of backing 120 as backing 120 travels in insertion direction 75 (as shown in FIG. 2B).

In particular, as shown in FIG. 2B, pawls 141A, 142A may contact teeth 121, 122 of sides 120A, 120B respectively. Each side 120A, 120B may comprise one or more teeth 121, 122. Each tooth 121, 122 may comprise a beveled reverse-direction face 121A, 122A (e.g. a surface having a normal vector with a component in reverse direction 80) and a beveled insertion-direction face 121C, 122C (e.g. a surface having a normal vector with a component in insertion direction 75). The beveled reverse-direction faces 121A, 122A and insertion-direction faces 121C, 122C of adjacent teeth 121, 122 may define a concavity 121B, 122B between the adjacent teeth 121, 122.

As pawls 141A, 142A contact teeth 121, 122 while backing 120 is moving in insertion direction 75, pawls 141A, 142A first contact beveled insertion-direction faces 121C, 122C. Insertion-direction faces121C, 122C may be beveled with bevel angles which cause pawls 141A, 142A to move away from each other (either by movement/deformation of pawls 141A, 142A or by movement/deformation of other elements locks 141, 142), thereby allowing backing 120 to continue to travel in insertion direction 75.

On the other hand, if a net force applied to backing 120 was directed in second direction 80, pawls 141A, 142A protrude into, and are lockingly engaged in, concavities 121B, 122B. When pawls 141A, 142A project into concavities 121B, 122B, reverse-direction faces 121A, 122A may be shaped (e.g. relative to the extension directions of pawls 141A, 142A) to prevent movement of backing 120 in reverse direction 80. Accordingly, pawls 141A, 142A allow backing 120 to travel in insertion direction 75 such that microneedle 125 may be inserted into the tissue and pawls 141A, 142A (in conjunction with teeth 121, 122) prevent backing 120 from travelling in reverse direction 80, thereby mitigating against bounce-back which could cause microneedle 125 to withdraw from the tissue.

Once microneedle 125 is inserted into the tissue as desired, the applied force in directions 85 on portions 141C, 142C of locks 141, 142 may be released, thereby allowing the biasing members to cause pawls 141A, 142A to move apart from one another as shown in FIG. 2C. As pawls 141A, 142A move apart from one another, pawls 141A, 142A withdraw from concavities 121B, 122B, thereby releasing locking mechanism 140 and allowing backing 120 to move freely (i.e. without interacting with locking mechanism 140). Free movement of backing 120 allows fluid to be injected through microneedle 125 via conduit 135 and for tissue to expand in reverse direction 80 (e.g. for wheal, W, to form) without interference by locking mechanism 140 on the reverse direction movement of backing 120.

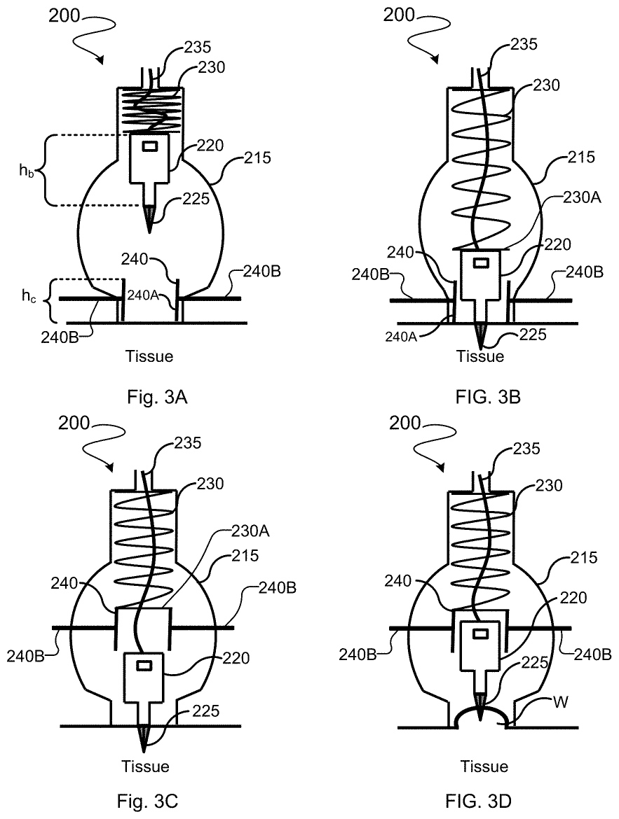

FIGS. 3A to 3D depict an apparatus 200 for inserting a microneedle into tissue according to a particular non-limiting embodiment of the invention. In the illustrated embodiment, apparatus 200 comprises a microneedle 225 supported by a backing 220. A forcer 230 is selectably operable between a retracted state (as depicted in FIG. 3A) and an extended state (as depicted in FIG. 3B). As forcer 230 transitions from the retracted state to the extended state, forcer 230 applies force against backing 220 in insertion direction 75 toward the tissue. Unlike the apparatus 10, 100 of FIGS. 1 and 2, forcer 230 does not “fire” backing 220 in insertion direction 75, but instead applies force to backing 220 until after microneedle 225 is inserted into the tissue and any bounce-back forces have subsided. A forcer withdrawal mechanism 240 may be employed to withdraw forcer 230 in reverse direction 80 away from the tissue after insertion of microneedle 225 into the tissue, after any bounce-back forces have subsided, but prior to injection of fluid through microneedle 225 into the tissue. Forcer withdrawal mechanism 240 may lockingly engage forcer 230 to prevent motion of forcer 230 in insertion direction 75 after withdrawal.

Similar to apparatus 10 described above, backing 220, microneedle 225 and/or at least some of forcer 230 may be contained in a housing 215. Housing 215 may be substantially similar to housing 15.

Forcer 230 may comprise any suitable forcer capable of applying force against backing 220 in direction 75. Forcer 230 may be substantially similar to forcer 30. Forcer 230 may comprise a store of potential energy wherein at least a portion of the potential energy stored in forcer 230 is released as forcer 230 operates from its retracted configuration to its extended configuration. In some embodiments, at least some of the potential energy of forcer 230 remains in the store after insertion of microneedle 225 in the tissue.

Apparatus 200 may differ from apparatus 10 in that forcer 230 does not “fire” backing 220 (as a projectile) in insertion direction 75. Instead, forcer 230 remains in contact with, and continues to apply force in insertion direction 75 to, backing 220 until, and for at least a period of time after, microneedle 225 is inserted into the tissue. In this way, forcer 230 may continue to apply force to backing 220 in insertion direction 75 to mitigate bounce-back of microneedle 225 upon impact with the tissue, thereby ensuring that microneedle 225 does not undesirably withdraw from the tissue in reverse direction 80.

Once microneedle 225 is inserted into the tissue as desired and after any associated bounce-back forces have subsided, forcer 230 may be disengaged from backing 220 by forcer withdrawal mechanism 240, as depicted in FIG. 3C. Forcer withdrawal mechanism 240 may comprise any suitable mechanism capable of moving forcer 230 in reverse direction 80 away from backing 220 or otherwise performing any of the functionality described herein.

For example, in the illustrated embodiment, forcer withdrawal mechanism 240 comprises a collar 240A. Collar 240A has an internal diameter that is greater than the external diameter of backing 220, so that collar 240A and backing 220 can move freely relative to one another in insertion direction 75 or reverse direction 80. Collar 240A may be co-axial or substantially co-axial with backing 220 such that collar 240A does not interfere with backing 220 as backing 220 travels in insertion direction 75 to insert microneedle 225 into the tissue as depicted in FIGS. 3A and 3B.

Collar 240A may have a height h_{c} that is less than a height h_{b} of backing 220 such that forcer 230 does not contact collar 240A when microneedle 225 is inserted into the tissue (see FIG. 3A). The internal diameter of collar 240A may be smaller than the external diameter of forcer 230 or forcer 230 may comprise a forcing plate 230A, such that when collar 240A is moved in reverse direction 80, it contacts forcer 230 and moves forcer 230 in reverse direction 80 to disengage forcer 230 from backing 220, as depicted in FIG. 3C.

Collar 240A may be caused to move in reverse direction 80 by applying force to one or more tabs 240B attached to collar 240A. In some embodiments, the tabs 240B may protrude through housing 215. Collar 240A may be moved in reverse direction 80 by applying force to tabs 240B, for example, manually, by magnetic force, by one or more motors, by one or more pulleys, by a wind-up mechanism, etc. Once collar 240A is moved in reverse direction 80 such that forcer 230 no longer engages backing 220, and backing 220 is free to move with the expansion of the tissue in reverse direction 80 (e.g. as a wheal is formed in response to injection of fluid through microneedle 225).

A locking mechanism may be provided to hold collar 240A in place after forcer 230 is moved in reverse direction 80 and disengaged from backing 220. The locking mechanism may comprise one or more protrusions or bumps that may deform (or that may be supported by a deformable material) such that collar 240A is allowed to engage the protrusions or bumps and is then prevented from moving back in insertion direction 75 by such protrusions or bumps. In some embodiments, tabs 240B of collar 240A be lockingly engaged by housing 215 (e.g. by rotation of collar 240A about the needle axis). In some embodiments, an actuator applies continuous force against collar 240A to prevent collar 240A from moving back in insertion direction 75. In some embodiments, an additional forcer is provided to restrain collar 240A. In some embodiments, collar 240A is restrained manually.

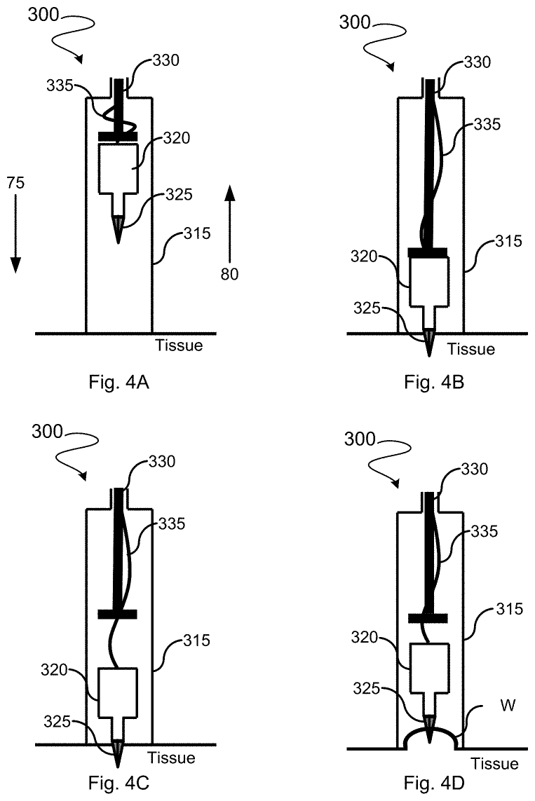

FIGS. 4A to 4D depict an apparatus 300 for inserting a microneedle into tissue according to a particular non-limiting embodiment of the invention. Apparatus 300 is substantially similar to apparatus 200. For example, apparatus 300 comprises a housing 315 similar to housing 215, a backing 320 similar to backing 220, a microneedle 325 similar to microneedle 225, a forcer 330 similar to forcer 230, a conduit 335 similar to conduit 235. Unlike apparatus 200, the forcer withdrawal mechanism of apparatus 300 is a feature of forcer 330 itself.

Forcer 330 may comprise any suitable forcer capable of applying force against backing 320 by extending in direction 75 and capable of withdrawing force from backing 320 by retracting in reverse direction 80. For example, forcer 330 may comprise an electromechanical forcer capable of moving in insertion direction 75 and in reverse direction 80, or may be manually operated. In this way, forcer 330 may apply force in insertion direction 75 against backing 320 and then disengage from backing 320 by moving in reverse direction 80, as needed.

Apparatus 300 may also be similar to apparatus 200 in that forcer 230 may remain in contact with, and continue to apply force in insertion direction against, backing 320 until and for at least a period of time after microneedle 325 is inserted into the tissue and any bounce-back forces have subsided. In this way, forcer 330 may continue to apply force to backing 320 in insertion direction 75 to reduce bounce-back of microneedle 325 upon impact with the tissue, thereby ensuring that microneedle 325 does not undesirably withdraw from the tissue.

Once microneedle 325 is inserted into the tissue as desired, forcer 330 may be disengaged from backing 320, as depicted in FIG. 4C. Apparatus 300, unlike apparatus 200 does not include a separate forcer withdrawal mechanism. Instead, forcer 330 is capable of withdrawing on its own, using for example an electromechanical mechanism, mechanical mechanism, pneumatic mechanism, hydraulic mechanism, or manually.

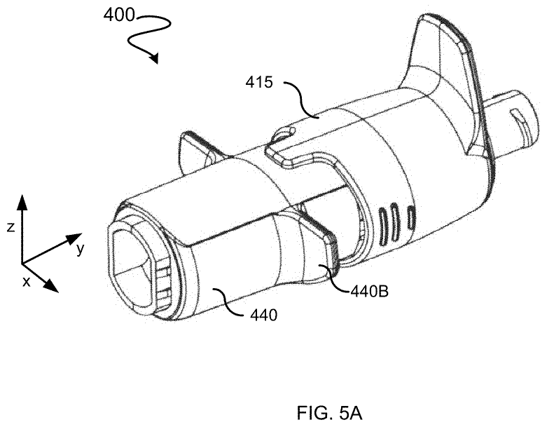

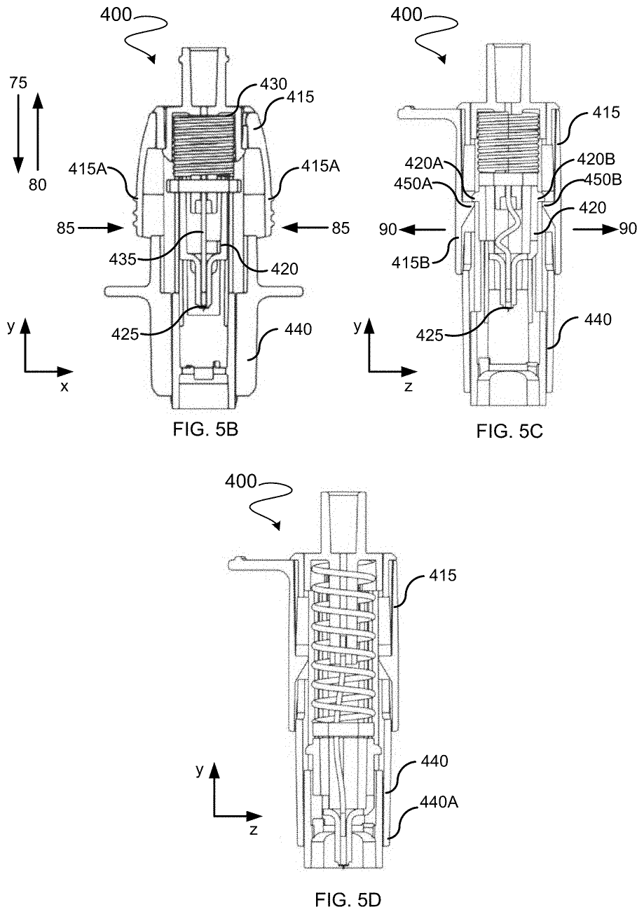

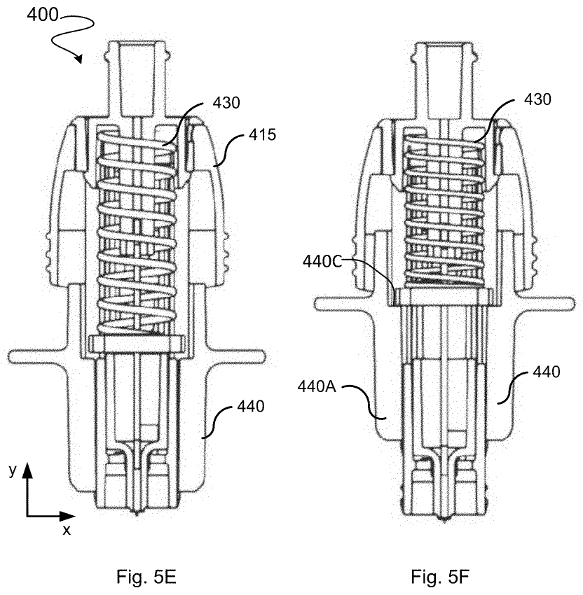

FIGS. 5A to 5F (collectively, FIG. 5) depict an apparatus 400 for inserting a microneedle into tissue according to a particular non-limiting embodiment of the invention. Apparatus 400 may be substantially similar to apparatus 200. For example, apparatus 400 comprises a housing 415 similar to housing 215, a backing 420 similar to backing 220, a microneedle 425 similar to microneedle 225, a forcer 430 similar to forcer 230, a conduit 435 similar to conduit 235, and forcer withdrawal mechanism 440 similar to forcer withdrawal mechanism 240.

As can be seen from FIG. 5B, forcer 430 of apparatus 400 comprises a spring. Forcer 430 abuts against backing 420 to apply force against backing 420 in insertion direction 75. However, backing 420 may be prevented from moving in insertion direction 75 by stops 450A, 450B (similar to stops 50), as best seen in FIG. 5C. In particular, flanges 420A, 420B of backing 420 abut against stops 450A, 450B, thereby preventing travel of backing 420 in insertion direction 75 (and maintaining forcer 430 in its retracted state).

To allow forcer 430 to go from its retracted state to its extended state and to apply force which causes backing 420 to move in insertion direction 75, force may be applied in X-axis transverse directions 85 to portion 415A of housing 415. As can be seen by comparing FIGS. 5B and 5C, housing 415 is shaped such that its cross-sectional shape in a plane defined by the X and Z directions (e.g. its transverse cross-sectional shape) is oval-like. By squeezing the long direction dimension (i.e. the X direction dimension) of the oval (e.g. by applying force in X-axis transverse directions 85 to portions 415A of housing 415), housing 415 may deform such that the oval-like shape becomes more circle-like and the short direction dimension (i.e. the Z-axis dimension) of the oval expands, thereby causing stops 450A, 450B to move apart from one another in Z-axis transverse directions 90. When stops 450A, 450B move apart from one another in Z-axis directions 90, flanges 420A, 420B lose contact with stops 450A, 450B and backing 420 is allowed to travel in insertion direction 75, as shown in FIG. 5D. Although housing 415 of the illustrated FIG. 5 embodiment has an oval-like transverse cross-sectional shape in the X-Z plane, other transverse cross-sectional shapes, such as but not limited to circular or rectangular shapes, may be employed with similar results.

In some embodiments, the deformation of housing 415 is elastic and after the forces applied to housing 415 in direction-axis transverse directions 85 are released, housing 415 restoratively deforms to (or close to) its original shape. This is not necessary. In other embodiments, housing 415 plastically deforms and might only be used once.

As forcer 430 extends in insertion direction 75, forcer 430 forces backing 420 in insertion direction 75 toward the tissue until microneedle 425 contacts and is inserted into the tissue, as depicted in FIG. 5E. Bounce-back of backing 420 in direction 80 is reduced or prevented completely due to the continuing force applied by forcer 430 on backing 420 in insertion direction 75 during insertion of microneedle 425 into the tissue and for a period of time after insertion of microneedle 425 into the tissue until any bounce-back forces have subsided.

Once microneedle 425 is inserted into the tissue as desired, forcer withdrawal mechanism 440 may be employed to disengage forcer 430 from backing 420. In the illustrated embodiment, forcer withdrawal mechanism 440 comprises a collar 440A that is translatable in insertion direction 75 and in reverse direction 80. Collar 440A is substantially similar to collar 240A described herein. Collar 440A comprises a pair of tabs 440B for applying force to collar 440A in reverse direction 80. Collar 440 comprises a shoulder 440C that can abut a portion of forcer 430. By applying force to collar 440A in reverse direction 80, shoulder 440C abuts forcer 430 and moves forcer 430 in reverse direction 80 to disengage forcer 430 from backing 420. Collar 440A may be held in place (e.g. prevented from moving back in insertion direction 75) by one or more features, such as those discussed in relation to collar 240A. By disengaging forcer 430 from backing 420, backing 420 and microneedle 425 become free to move in directions 75, 80 as the tissue expands or contracts due to the injection of fluid via conduit 435 and the corresponding formation of a wheal or due to the extraction of matter from the tissue.

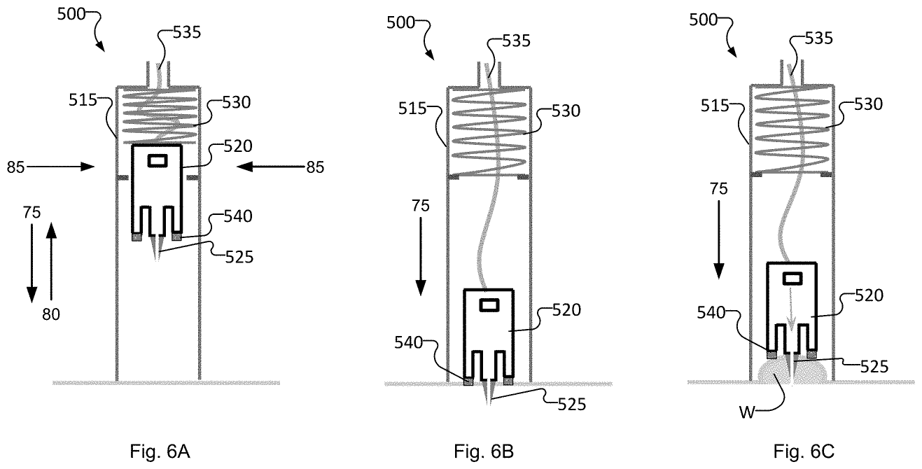

FIGS. 6A to 6C depict an apparatus 500 for inserting a microneedle into tissue according to a particular non-limiting embodiment of the invention. Apparatus 500 is substantially similar to apparatus 100. For example, apparatus 500 comprises a housing 515 similar to housing 15, a backing 520 similar to backing 20, a microneedle 525 similar to microneedle 25, a forcer 530 similar to forcer 30, a conduit 535 similar to conduit 35. To allow forcer 530 to go from its retracted state to its extended state and to apply force which causes backing 520 to move in insertion direction 75, force may be applied in transverse directions 85 to a portion housing 515. Like apparatus 100, forcer 530 of apparatus 500 “fires” backing 520 (e.g. as a projectile) in insertion direction 75 and backing 520 disengages from forcer 530 and continues to travel (e.g. as a projectile) in insertion direction 75 toward the tissue. Unlike apparatus 100, apparatus 500 comprises one or more adhesive surfaces 540 instead of a locking mechanism 40.

Adhesive surface 540 may be a surface of backing 520 or may be attached to backing 20 in any suitable manner such that adhesive surface 540 contacts the tissue when microneedle 525 is inserted into the tissue, as shown in FIG. 6B. As backing 520 travels (e.g. as a projectile) in insertion direction 75 toward the tissue, adhesive surface(s) 540 may engage (e.g. adhesively stick to or adhere to) the tissue to mitigate against bounce-back.

Adhesive surface(s) 540 may surround or partially encircle (although this does not require that adhesive surface(s) 540 are circular in shape) microneedle 525 (e.g. in a plane extending into and out of the page in FIGS. 6A-6C). The adhesive material of adhesive surface(s) may comprise any suitable adhesive such as, but not limited to, solvent-based adhesives, polymer dispersion adhesives, pressure-sensitive adhesives, contact adhesives, multi-component adhesives, one-part adhesives, and natural adhesives.

After microneedle 525 is inserted into the tissue as desired, microneedle 525 may be used to, for example, inject fluid into the tissue, sense and/or monitor interstitial fluid (ISF), blood, skin composition, etc., apply electrical current to the tissue, extract matter from the tissue etc. In the FIG. 6 embodiment, fluid may be injected into the tissue via conduit 535. As fluid is injected into the tissue, the tissue may begin to expand (e.g. the tissue may form a wheal, W). Since forcer 530 is disengaged from backing 520 and adhesive surface(s) 540 provide no insertion direction 75 force on backing 20, the tissue is allowed to expand without interference from backing 520.

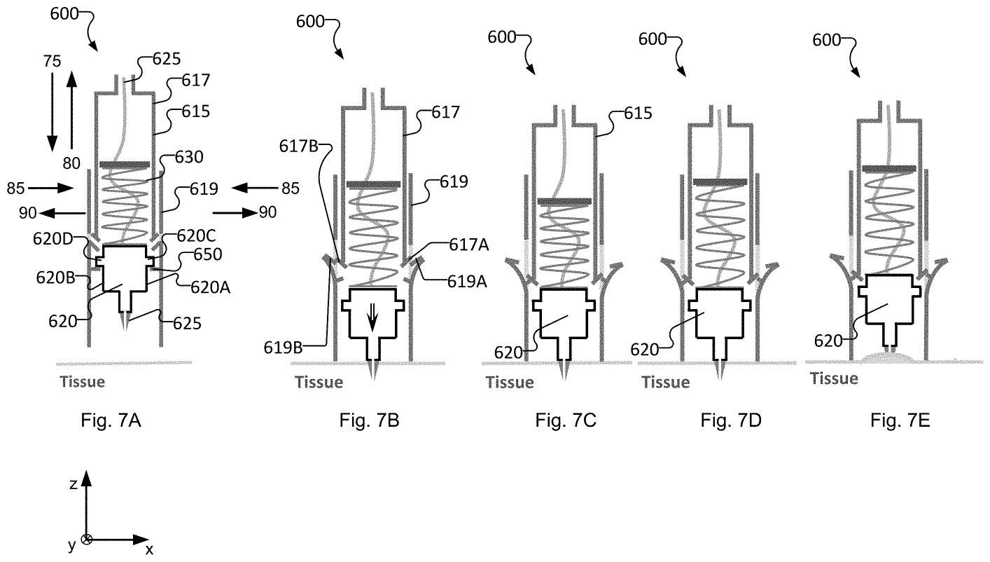

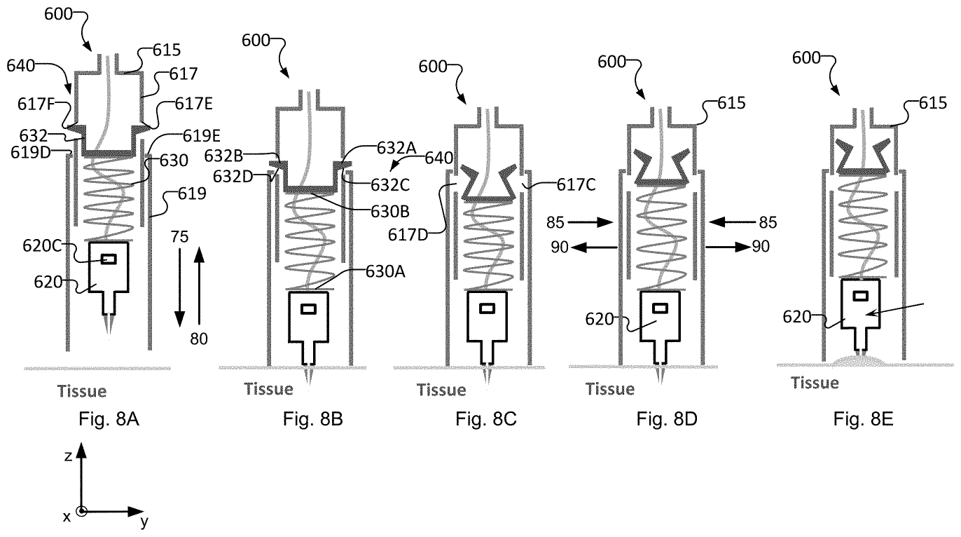

FIGS. 7A to 7E depict an apparatus 600 for inserting a microneedle into tissue according to a particular non-limiting embodiment of the invention. FIGS. 8A to 8E depict the apparatus of FIGS. 7A to 7E from a second perspective (e.g. rotated by 90° about the Z—axis). It should be understood that reference to the “FIG. 7 embodiment” is also meant to include the features of FIG. 8 as FIG. 8 also depicts the “Figure 7 embodiment” (e.g. apparatus 600). Apparatus 600 is substantially similar to apparatus 200 except as follows. For example, apparatus 600 comprises a housing 615 similar to housing 215, a backing 620 similar to backing 220, a microneedle 625 similar to microneedle 225, a forcer 630 similar to forcer 230, a conduit 635 similar to conduit 235. Unlike apparatus 200, forcer 630 is not withdrawn by a forcer withdrawal mechanism but is instead released by a release mechanism 640.

Forcer 630 is selectably operable between a retracted state (as depicted in FIG. 7A) and an extended state (as depicted in FIG. 7B). As compared to the extended state, when forcer 630 is in its retracted state, a proximate end 630A of forcer 630 is spaced apart from a distal end 630B of forcer 630, opposite the proximate end 630A of forcer 630, by a relatively small distance—i.e. in its extended state, the distance between the proximate end 630A and distal end 630B of forcer 630 is larger than the distance between the proximate end 630A and distal end 630B of forcer 630 when forcer is in its retracted state. As forcer 630 transitions from the retracted state to the extended state, proximate end 630A of forcer 630 applies force against backing 620 in insertion direction 75 toward the tissue. Unlike apparatus 10 and apparatus 100 of FIGS. 1 and 2, forcer 630 does not “fire” backing 620 in insertion direction 75, but instead applies force to backing 620 until after microneedle 625 is inserted into the tissue and any bounce-back forces have subsided. A release mechanism 640 may be employed to release forcer 630 to remove or reduce force applied by a proximate end 630A of forcer 630 to backing 620 after insertion of microneedle 625 into the tissue, after any bounce-back forces have subsided, but prior to injection of fluid through microneedle 625 into the tissue.

Forcer 630 may comprise any suitable forcer capable of applying force against backing 620 by extending in direction 75 and capable of being released, as discussed further below. For example, in the depicted embodiment, forcer 630 comprises a spring.

Forcer 630 may be naturally biased toward its extended state and may be held in the retracted state by one or more stops or stop-mechanisms (also referred to as a “catch”). For example, in the FIG. 7 embodiment, forcer 630 may be naturally biased toward its extended state (e.g. the spring of forcer 630 may be compressed) but forcer 630 may be held in its retracted state by one or more stops 650. In the FIG. 7 embodiment, stops 650 may physically engage (e.g. abut) backing 620 to prevent backing 620 from moving in insertion direction 75 or to otherwise prevent forcer 630 from applying force which would tend to move backing 620 in insertion direction 75. When stops 650 engage backing 620 (as is the case in FIG. 7A), backing 620 in turn prevents forcer 630 from activating or extending. By releasing stops 650, forcer 630 may be free to activate or expand and may apply force which tends to move backing 620 in insertion direction 75. In some embodiments, stops 650 may additionally or alternatively engage forcer 630 directly to prevent backing 620 from moving in insertion direction 75 or to otherwise prevent forcer 630 from applying force which would tend to move backing 620 in insertion direction 75.

Stops 650 may comprise any suitable stopping mechanism. In some embodiments, stops 650 comprise one or more protrusions or flanges extending from an interior surface of housing 615 (or another part of apparatus 600). Stops 650 may physically engage (e.g. abut against) backing 620 and/or forcer 630. Stops 650 may be physically disengaged from backing 620 or forcer 630 using any suitable technique. In the illustrated embodiment of FIG. 7A, stops 650 may be disengaged from backing 620 or forcer 630 by deforming (either elastically or plastically) at least a portion of housing 615. In some embodiments, stops 650 may comprise one or more moveable parts releasable by pivoting, sliding, ratcheting, twisting, compressing or the like. Stops 650 may comprise one or more solenoid actuators or the like. In some embodiments, stops 650 may physically engage a flange (not shown) of backing 620 and/or sides 620A, 620B of backing 620. In some embodiments, stops 650 engage one or more teeth or protrusions 620C, 620D on sides 620A, 620B of backing 620.

In the FIG. 7A embodiment, stops 650 are released from backing 620 by applying force to housing 615 in a direction parallel to insertion direction 75. Housing 615 comprises a first section 619 and a second section 619. At least a portion of first section 617 is slidably receivable in second section 619. First and second sections 617, 619 may be nested tubes having circular, ovoid, rectangular etc. cross-sections in a plane defined by the X and Y directions (e.g. an XY-plane). In some embodiments, first and second sections 617, 619 may be concentric, although this is not mandatory. In the illustrated embodiment, first section 617 comprises a beveled surface 617A and second section 619 comprises a beveled surface 619A. Beveled surface 617A may be beveled so as to face at least partially in a radially outward direction 90 and at least partially in insertion direction 75 while beveled surface 619B may be beveled so as to face at least partially in a radially inward direction 85 and at least partially in reverse direction 80. A force may be applied to first section 617 in a direction parallel to insertion direction 75 to cause first section 617 to move in insertion direction 75 relative to second section 619 thereby causing beveled surface 617A to contact beveled surface 619A. As beveled surface 617A contacts beveled surface 619A, beveled surface 617A applies a force in radially outward direction 90 to beveled surface 619A causing at least a portion of second section 619 to deform in the radially outward direction 90. Such deformation may be elastic or plastic. As second section 619 deforms radially outwardly (e.g. in radial outward direction 90), stops 650 may also move radially outwardly until stops 650 disengage protrusions 620C, 620D of backing 620. When stops 650 disengage from protrusions 620C, 620D of backing 620, forcer 630 applies force which tends to move backing 620 in insertion direction 75 past stops 650. In some embodiments, deformation of second section 619 is elastic and restorative deformation of second section 619 (e.g. deformation returning second section 619 to its non-deformed shape or close thereto) occurs after the force on second section 619 is relieved (e.g. after beveled surfaces 617A, 617B move past beveled surfaces 619A, 619B in insertion direction 75). This is not mandatory.

While the FIG. 7 embodiment depicts stops 650 as protruding from an inner surface of second section 619, this is not mandatory. In other embodiments, stops 650 could, for example, protrude from an inner surface of first section 617 and instead of first section 617 being slidably receivable in second section 619, it may be that second section 619 is slidably receivable in first section 617 and it is first section 617 that is deformed to disengage stops 650 from backing 620. In some embodiments, instead of housing 615 deforming, a portion of backing 620 may deform to release stops 650.

In other embodiments, backing 620 may be released from stops 650 by rotating backing 620 relative to second section 619 of housing 615. For example, backing 620 may be configured such that if backing 620 is rotated sufficiently in relation to section portion 619, stops 650 no longer engage backing 620 and backing 620 is free to move relative to second section 619 of housing 615. In some embodiments, backing 620 is rotated relative to second section 619 of housing 615 by rotating first section 617 of housing 615. In some embodiments, insertion direction 75 movement of first section 617 relative to second section 619 may cause rotation of backing 620 due to, for example, abutment of one or more beveled surfaces of backing 620 with one or more beveled surfaces of first section 617 and/or second section 619.

Apparatus 600 may also be similar to apparatus 200 in that forcer 630 may remain in contact with, and continue to apply force in insertion direction against, backing 620 until and for at least a period of time after microneedle 625 is inserted into the tissue and any bounce-back forces have subsided. In this way, forcer 630 may continue to apply force to backing 620 in insertion direction 75 to reduce bounce-back of microneedle 625 upon impact with the tissue, thereby ensuring that microneedle 625 does not undesirably withdraw from the tissue.

Once microneedle 625 is inserted into the tissue as desired, forcer 630 may be released. In some embodiments, releasing forcer 630 may comprise disengaging a proximal end 630A of forcer 630 from backing 620. In other embodiments, releasing forcer 630 may comprise releasing a distal end 630B of forcer 630. For example, in the FIG. 7 embodiment, distal end 630B of forcer 630 is released by a release mechanism 640. Release mechanism 640 may release distal end 630B of forcer 630 by releasing a forcer support 632 relative to housing 615 to allow forcer support 632 and/or distal end of forcer 630 to move in reverse direction 80 relative to housing 615 such that the distance between proximate end 630A of forcer 630 and distal end 630B of forcer 630 may increase due to the bias of forcer 630 or otherwise. In some embodiments, the distance between proximate end 630A of forcer 630 and distal end 630B of forcer 630 may be allowed to increase sufficiently for forcer 630 to be in its extended state such that forcer 630 does not apply force (aside gravitational force) to backing 620 when forcer support 632 is released. In this way, when forcer 630 is released by release mechanism 640, backing 620 and microneedle 625 in turn may be allowed to move relatively freely in reverse direction with the tissue.

Referring now to FIG. 8A, while proximal end 630A of forcer 630 engages (e.g. abuts) backing 620, distal end 630B of forcer 630 engages (e.g. abuts) a forcer support 632. During insertion of microneedle 625 into the tissue, forcer support 632 is fixed relative to first section 617 of housing 615 such that whether backing 620 is engaged or disengaged by stops 650, distal end 630B of forcer 630 remains fixed relative to first section 617. In some embodiments, forcer support 632 is releasable relative to first section 617 of housing 615.

In the FIG. 7 embodiment, forcer support 632 comprises first and second flanges 632A, 632B which are projectable into first and second slots 617C, 617D, as shown in FIG. 8A. This is not mandatory. For example, support 632 could define slots while section 617 could comprise protrusions which project into such slots. In some embodiments, first and second flanges 632A, 632B are biased in radial outward direction 90 (e.g. toward or into first and second slots 617C, 617D when first and second flanges 632A, 632B are aligned with first and second slots 617C, 617D). Such bias may be caused by, for example, the elasticity of the material of forcer support 632 or an alternative mechanism such as a spring. When first and second flanges 632A, 632B of forcer support 632 project into first and second slots 617C, 617D, relative movement in the forward direction 75 and/or reverse direction 80 between forcer support 632 and housing 615 is prevented by the interaction between first and second flanges 632A, 632B of forcer support 632 and first and second slots 617C, 617D.

First and second flanges 632A, 632B may be forced out of slots 617C, 617D in a radially inward direction 85 to allow relative movement in the forward direction 75 and/or reverse direction 80 between forcer support 632 and housing 615 to thereby release forcer 630. Forcing first and second flanges 632A, 632B out of slots 617C, 617D may comprise deforming support 632 or pivoting one or more portions of support 632 (not depicted). In some embodiments, first and second flanges 632A, 632B are manually forced out of first and second slots 617C, 617D in a radially inward direction by applying a force to each of first and second flanges 632A, 632B in radially inward direction 85. In other embodiments, applying force to first section 617 of housing 615 in the forward direction 75 may cause first and second flanges 632A, 632B to move in radial inward direction 85 and out of first and second slots 617C, 617D. In some embodiments, first and second flanges 632A, 632B may be disengaged from first and second slots 617C, 617D if force applied to housing 615 in insertion direction 75 after backing 620 disengages from stops 650. In this way, a single application of force to first section 617 may cause backing 620 to disengage from stops 650 (to thereby allowing forcer 630 to cause microneedle 625 to penetrate the tissue) and may cause flanges 632A, 632B to disengage slots 617C, 617D (to thereby release forcer 630 and allow backing 620 to move with the tissue and/or allow a wheal to form).

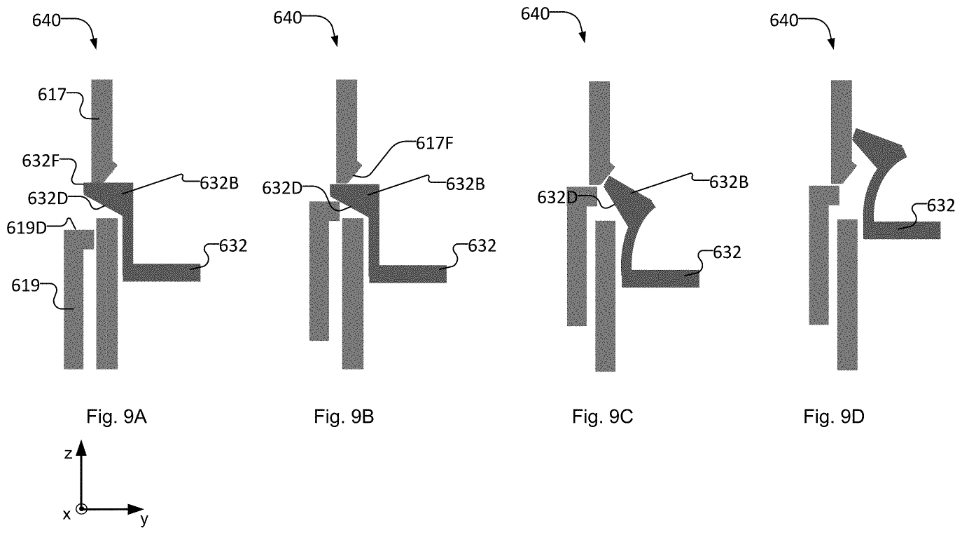

FIGS. 9A to 9D depict an expanded portion of release mechanism 640. In the depicted embodiment, flanges 632A, 632B comprise beveled surfaces 632C, 632D. Beveled surfaces 632C, 632D may be beveled so as to face at least partially in radially outward direction 90. As first section 617 moves in forward direction 75 relative to second section 619, flanges 632A, 632B may contact surfaces 619C, 619D of second section 619 thereby causing flanges 632A, 632B to move in radially inward direction 85 (as can be seen in FIGS. 9B and 9C). Such movement may comprise, for example, deformation of at least a portion of forcer support 632 or pivoting of one or more pivotable arms of support 632 attached to flanges 632A, 632B (not depicted). As first section 617 continues to move in forward direction 75 relative to second section 619 tips 632E, 632F of flanges 632A, 632B are forced against edges 617E, 617F of slots 617C, 617D, thereby causing flanges 632A, 632B to move radially inwardly and out of slots 617C, 617D as shown in FIGS. 9C and 9D.

Once flanges 632A, 632B move radially inwardly and out of slots 617C, 617D, reverse direction 80 force applied by distal end 630B of forcer 630 may cause support 632 to continue to move in reverse direction 80 relative to first section 617 of housing 615. In some embodiments, to guide movement of flanges 632A, 632B in radially inward direction 85 while support 632 continues to move in reverse direction 80 relative to first section 617 of housing 615, edges 617E, 617F may be beveled so as to face at least partially in a radially inward direction 85. As first and second flanges 632A, 632B abut against edges 617E, 617F due to the reverse direction 80 forcer of forcer 630, first and second flanges 632A, 632B may be caused to move radially inwardly until first and second flanges 632A, 632B disengage (e.g. cease to abut) edges 617E, 617F. Such movement of flanges 632A, 632B may comprise, for example, deformation of at least a portion of forcer support 632 or pivoting of one or more pivotable arms of support 632 attached to flanges 632A, 632B (not depicted).

In other embodiments, flanges 632A, 632B may be released from slots 617C, 617D by rotating first section 617 of housing 615 relative to second section 619 of housing 615.

For example, in some embodiments, a portion of second section 619 of housing 615 may prevent forcer support 632 from rotating relative to second section 619 which may cause flanges 632A, 632B to rotate (relative to first section 617C) out of slots 617C, 617D when first section 617 of housing 615 is rotated relative to second section 619 of housing 615. Flanges 632A, 632B may be beveled to facilitate such movement. In some embodiments, insertion direction 75 movement of first section 617 relative to second section 619 may cause backing support 632 to rotate (e.g. due to abutting beveled surfaces of support plate 632 and first and/or second sections 617, 619) which may cause flanges 632A, 632B to rotate out of slots 617C, 617D.

When flanges 632A, 632B are disengaged from slots 617C, 617D, forcer support 632 is free to move in reverse direction 80, thereby allowing forcer 630 to achieve its extended state, reduce the force applied to backing 620 and allow backing 620 to move with the tissue and/or a wheal as it forms, as shown in FIGS. 8C to 8F. Free movement of backing 620 allows fluid to be injected through microneedle 625 via conduit 635 and for tissue to expand in reverse direction 80 (e.g. for a wheal to form) without substantial interference by forcer 630 on reverse direction 80 movement of backing 620.

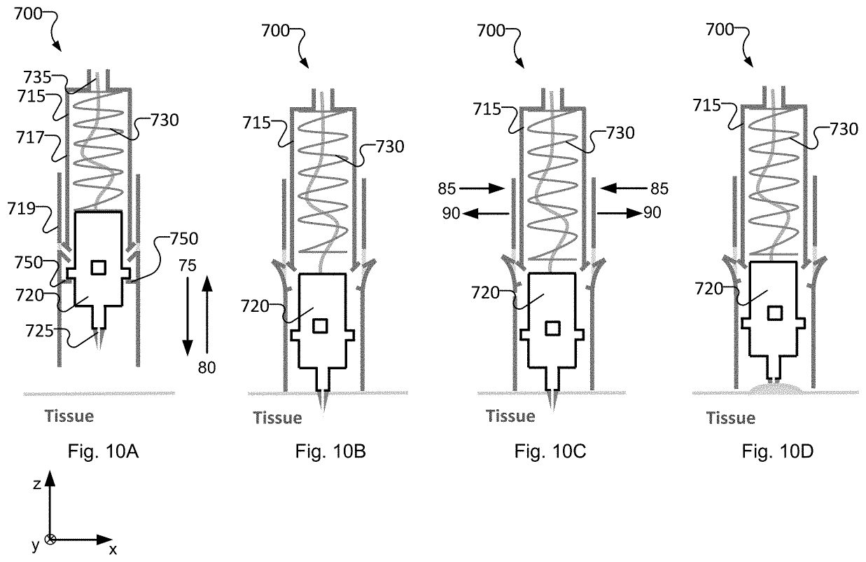

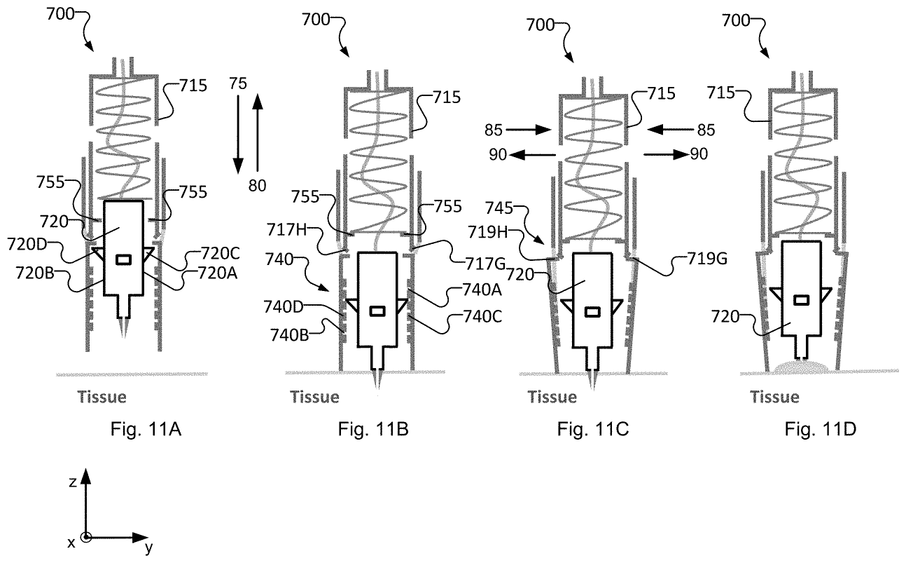

FIGS. 10A to 10D depict an apparatus 700 for inserting a microneedle into tissue according to a particular non-limiting embodiment of the invention. FIGS. 11A to 11D depict the apparatus of FIGS. 10A to 10D from a second perspective (e.g. rotated by 90° about the Z—axis). It should be understood that reference to the “Figure 10 embodiment” is also meant to include the features of FIG. 11, which also depicts the “Figure 10 embodiment” (e.g. apparatus 700). Apparatus 700 is substantially similar to apparatus 10 except as follows. For example, apparatus 700 comprises a housing 715 similar to housing 15, a backing 720 similar to backing 20, a microneedle 725 similar to microneedle 25, a forcer 730 similar to forcer 30, a conduit 735 similar to conduit 35.

Like the FIG. 1 embodiment, forcer 730 is naturally biased toward its extended state and is held in the retracted state by one or more stops or stop-mechanisms 750. However, unlike the FIG. 1 embodiment, stops 750 may be released by applying force to housing 715 in insertion direction 75. In this way, stops 750, housing 715 and backing 720 may be similar to stops 650, housing 615 and backing 620 for the purposes of holding forcer 730 in its retracted state and releasing stops 750 to allow forcer 730 to activate or extend to its extended state, as can be seen by comparing FIGS. 10A through 10D to FIGS. 7A through 7E. Like housing 615, housing 715 comprises a first section 717 and a second section 719. At least a portion of first section 717 is slidably receivable in second section 719. First and second sections 717, 719 may be nested tubes having circular, ovoid, rectangular etc. cross-sections in a plane defined by the X and Y directions (e.g. an XY-plane). In some embodiments, first and second sections 717, 719 may be concentric, although this is not mandatory.

As in the FIG. 1 embodiment, once stops 750 have released backing 720 and/or forcer 730 has been activated, forcer 730 applies force against backing 720 in insertion direction 75, thereby causing backing 720 (and microneedle 725) to move in insertion direction 75 toward the tissue. In the FIG. 10 embodiment, as forcer 730 extends, proximate end 730A of forcer contacts blocks 755 and the extent of its extension is limited (or as the extent of extension of forcer 730 is otherwise limited), backing 720 disengages from forcer 730 and continues to travel (e.g. as a projectile) in insertion direction 75 toward the tissue. Blocks 755 may comprise any suitable stopping mechanism for limiting the extension of forcer 730. For example, blocks 755 could comprise a ridge, a flange, or one or more protrusions protruding from an inner surface of a first section 717 of housing 715 as depicted in FIGS. 11A to 11D.

As backing 720 travels in insertion direction 75 toward the tissue, locking mechanism 740 may engage backing 720 to mitigate against bounce-back. In the illustrated embodiment of FIG. 10, as backing 720 travels in insertion direction 75 toward the tissue, backing 720 may pass from an unlocked configuration where backing 720 is not engaged by locking mechanism 740 (as depicted in FIG. 11A) to a locked configuration where backing 720 is engaged by locking mechanism 740 (as depicted in FIG. 11 B). As can be seen from FIGS. 11A and 11 B, backing 720 and locking mechanism 740 are in the unlocked configuration when backing 720 is relatively spaced apart from the tissue (in insertion direction 75) and are in the locked configuration when backing is relatively close (in insertion direction 75) to the tissue. In the unlocked configuration, backing 720 is free to move without such motion being influenced by locking mechanism 740. In the locked configuration, locking mechanism 740 lockingly engages backing 720 to prevent motion of backing 720 in reverse direction 80 opposed to insertion direction 75, while still allowing motion of backing 720 in insertion direction 75 toward the tissue. Locking mechanism 740 may therefore reduce bounce-back while allowing for backing 720 (and microneedle 725) to travel at sufficient velocity to ensure penetration of the tissue by microneedle 725.

Locking mechanism 740 may comprise any suitable locking mechanism for effecting such one-way motion of backing 720 and microneedle 725. In some embodiments like the FIG. 11 embodiment, locking mechanism 740 comprises at least one projection lockingly engageable in at least one concavity. In the FIG. 11 embodiment, side 720A of backing 720 comprises at least one prong 720C engageable by one or more concavities 740A of locking mechanism 740 and side 720B of backing 20 comprises at least one prong 720D engageable by one or more concavities 740B of mechanism 740. In some embodiments, concavities 740A, 740B may be located to engage the corresponding prongs 720C, 720D.

The concavities 740A, 740B of locking mechanism 740 may be formed by one or more teeth 740C, 740D on an inner surface of second section 719 of housing 715. To allow backing 720 to travel in insertion direction 75, surfaces of the prongs 720B, 720C that face at least partially in insertion direction 75 (e.g. have normal vectors with components in insertion direction 75) may be beveled such that the teeth 740C, 740D slide relatively easily (e.g. without significant decelerating force in reverse direction 80) along the beveled portion of the prongs 720C, 720D as backing 720 travels in insertion direction 75. In some embodiments, such motion of backing 720 in insertion direction 75 may comprise deformation of one or more of prongs 720C, 720D, sides 720A, 720B of backing 720, teeth 740C, 740D of locking mechanism 740 and second section 719 of housing 715. In some embodiments, the bevel angle of the surface of teeth 740C, 740D may additionally or alternatively be shaped to permit motion of backing 720 relative to teeth 740C, 740D in insertion direction 75.

On the other hand, to prevent backing 720 from travelling in reverse direction 80 opposed to insertion direction 75, surfaces of prongs 720C, 720D that face at least partially in reverse direction 80 (e.g. have normal vectors with components in reverse direction 80) may be beveled such that prongs 720C, 720D are guided into concavities 740A, 740B and backing 720 is incapable of sliding relative to teeth 740C, 740D to travel in reverse direction 80. In some embodiments, the bevel angle of the surface of teeth 740C, 740D may additionally or alternatively be shaped to guide prongs 720C, 720D into concavities 740A, 740B and prevent motion of backing 720 prongs 720C, 720D in reverse direction 80. In the locked configuration, locking mechanism 740 permits motion of at least a portion of backing 720 in insertion direction 75 from a first side one or more teeth 740C, 740D to a second side of the one or more teeth 740C, 740D and interaction between one or more teeth 740C, 740D and backing 720 prevents motion of the at least a portion of backing 720 in reverse direction 80 from the second side of one or more teeth 740C, 740D to the first side of the one or more teeth 740C, 740D.

In practice, as forcer 730 extends from its retracted configuration (FIG. 11A) to its extended configuration (FIG. 11 B), backing 720 is caused to move in insertion direction 75 toward the tissue by the application of force by forcer 730. In the illustrated FIG. 11 embodiment, forcer 730 encounters blocks 755 which prevent further extension of forcer 730, thereby causing forcer 730 to “fire” backing 720 as a projectile. Backing 720 continues to move in insertion direction 75 until microneedle 725 is inserted into the tissue. Microneedle 725 is prevented from withdrawing from the tissue (in the reverse direction 80) by locking engagement of locking mechanism 740 with backing 720. Accordingly, bounce-back of microneedle 725 out of the tissue is reduced as compared to other methods and apparatus.

After microneedle 725 is inserted into the tissue as desired, microneedle 725 may be used to, for example, inject fluid into the tissue, apply electrical current to the tissue, extract matter from the tissue etc. In the FIG. 11 embodiment, fluid may be injected into the tissue via conduit 735. As fluid is injected into the tissue, the tissue may begin to expand (e.g. the tissue may form a wheal). To allow the tissue to expand without interference from backing 720, locking mechanism 740 may be released before injection of fluid into the tissue.

Locking mechanism 740 may be released by any suitable mechanism or using any suitable method. In some embodiments, a locking mechanism release 745 allows locking mechanism 740 to be released by applying force to housing 715 in insertion direction 75, to thereby cause at least a portion of housing 715 to deform (elastically or plastically) disengage prongs 720C, 720D from concavities 740A, 740B and teeth 740C, 740D to allow reverse direction 80 movement of backing 720.

Locking mechanism release 745 may function in a substantially similar way to how stops 650, 750 are released. For example, In the FIG. 11A embodiment, locking mechanism 740 is released from backing 720 by applying force to housing 715 in a direction parallel to insertion direction 75. In the illustrated embodiment, first section 717 comprises beveled surfaces 717G, 717H and second section 719 comprises beveled surfaces 719G, 719H. Beveled surfaces 717G, 717H may be beveled so as to face at least partially in a radially outward direction 90 and at least partially in insertion direction 75 while beveled surfaces 719G, 719H may be beveled so as to face at least partially in a radially inward direction 85 and at least partially in reverse direction 80. A force may be applied to first section 717 in a direction parallel to insertion direction 75 to cause first section 717 to move in insertion direction 75 relative to second section 719 thereby causing beveled surfaces 717G, 717H to contact beveled surfaces 719G, 719H. As beveled surfaces 717G, 717H contact beveled surfaces 719G, 719H, beveled surfaces 717G, 717H apply force in radially outward direction 90 to beveled surfaces 719G, 719H causing at least a portion of second section 719 to deform in the radially outward direction 90. Such deformation may be elastic or plastic. As second section 719 deforms radially outwardly (e.g. in radial outward direction 90), concavities 740A, 740B and teeth 740C, 740D may also move radially outwardly until prongs 720C, 720D disengage concavities 740A, 740B and teeth 740C, 740D of locking mechanism 740. When prongs 720C, 720D disengage concavities 740A, 740B and teeth 740C, 740D of locking mechanism 740, locking mechanism 740 is released from backing 720 and backing 720 is free to move in reverse direction 80 as the tissue expands, and/or as a wheal forms. In some embodiments, deformation of second section 719 is elastic and restorative deformation of second section 719 (e.g. deformation returning second section 719 to its non-deformed shape or close thereto) occurs after the force on second section 719 is relieved (e.g. after beveled surfaces 717G, 717H move past beveled surfaces 719G, 719H in insertion direction 75). This is not mandatory.

In other embodiments, locking mechanism 740 may be released by rotating first section 717 relative to second section 719 and/or by rotation backing 720 relative to second section 719. In some embodiments, backing 720 may be configured such that if backing 720 is rotated sufficiently in relation to section portion 719, prongs 720C, 720D no longer engage 740A, 740B and teeth 740C, 740D of locking mechanism 740 and backing 720 is free to move relative to second section 719. In some embodiments, applying force to first section 717 in insertion direction 75 may cause backing 720 to rotate relative to second section 719 thereby releasing backing 720 from locking mechanism 740. In such embodiments, backing 720 and one or more of first and second sections 717, 719 may comprise abuttable beveled portions that are arranged to translate linear motion of first section 717 relative to second section 719 into rotational motion of backing 720 and prongs 720C, 720D.

Once locking mechanism 740 is released, backing 720 is permitted to move in reverse direction 80. Fluid may be injected into the tissue and the tissue may be allowed to expand in reverse direction 80 (e.g. form a wheal), as depicted in FIG. 11 D, without being impeded by backing 720 or locking mechanism 740.

Where a component is referred to above, unless otherwise indicated, reference to that component (including a reference to a “means”) should be interpreted as including as equivalents of that component any component which performs the function of the described component (i.e. that is functionally equivalent), including components which are not structurally equivalent to the disclosed structure which performs the function in the illustrated exemplary embodiments of the invention.

Unless the context clearly requires otherwise, throughout the description and any accompanying claims (where present), the words “comprise,” “comprising,” and the like are to be construed in an inclusive sense, that is, in the sense of “including, but not limited to.” As used herein, the terms “connected,” “coupled,” or any variant thereof, means any connection or coupling, either direct or indirect, between two or more elements; the coupling or connection between the elements can be physical, logical, or a combination thereof. Additionally, the words “herein,” “above,” “below,” and words of similar import, shall refer to this document as a whole and not to any particular portions. Where the context permits, words using the singular or plural number may also include the plural or singular number respectively. The word “or,” in reference to a list of two or more items, covers all of the following interpretations of the word: any of the items in the list, all of the items in the list, and any combination of the items in the list.

As will be apparent to those skilled in the art in the light of the foregoing disclosure, many alterations and modifications are possible in the practice of this invention without departing from the spirit or scope thereof. For example:
The individual features of each of the embodiments herein can be interchanged. For example, the locking mechanism of one embodiment could be interchanged with the locking mechanism of another embodiment. Similarly, the stops or the mechanism to release the stops of one embodiment could be interchanged with the stops or the mechanism to release the stops of another embodiment.
Where a first component (e.g. a backing) is described herein as comprising one or more male member(s) (e.g. a prong, a projection, or a pawl) and a second component (e.g. a housing) comprises one or more complementary female member(s) (e.g. a concavity/teeth or a slot), the male and female members can be interchanged such that the first component (e.g. the backing) comprises the female member(s) and the second component (e.g. the housing) comprises the male member(s).
Where a first component or portion of the first component is described herein as being deformable upon contact with a second component or portion of the second component, it should be understood that the second component or portion of the second component could additionally or alternatively be deformable upon contact with the first component or portion of the first component.
Where embodiments herein are discussed in relation to inserting fluid via a microneedle, it should be understood that such embodiments and/or other embodiments could be used for extraction of fluids or other materials from the tissue, tissue sensing (e.g. sensing and monitoring interstitial fluid (ISF), blood, skin composition), housing other sensors for various tissue characteristics, combinations of these applications and/or the like.
While the apparatus described herein may have been described as stand-alone apparatus, the apparatus described herein could also be attached to, attachable to, combined with, or combinable with other apparatus such as syringes, sensors, reservoirs or the like.
While the description discusses releasing or allowing movement of the backing in a reverse direction 80 to accommodate formation of a wheal when fluid is injected into tissue, the apparatus should not be limited to allowing movement of the backing only in the reverse direction. In general, the application of force to the backing may be relieved or reduced to allow the backing to travel with the skin in any direction for the purposes of other applications such as fluid extraction, tissue sensing (e.g. sensing and monitoring interstitial fluid (ISF), blood, skin composition) etc. For example, the application of force to the backing may be relieved or reduced to allow the tissue to relax, contract, etc.
Many embodiments and variations are described above. Those skilled in the art will appreciate that various aspects of any of the above-described embodiments may be incorporated into any of the other ones of the above-described embodiments by suitable modification.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. An apparatus for inserting a microneedle into tissue, the apparatus comprising:
a microneedle supported by a backing to move therewith;
a forcer selectably operable between a retracted state and an extended state and wherein, upon operation of the forcer from the retracted state to the extended state, the forcer applies force to the backing which causes the backing to travel in an insertion direction toward the tissue;
a releasable locking mechanism which, in a locking state, permits one-way motion of the backing in the insertion direction toward the tissue and lockingly engages the backing to prevent motion of the backing in a reverse direction opposed to the insertion direction, the releasable locking mechanism releasable, to a released state, which permits motion of the backing in the reverse direction; and
wherein the forcer is disengaged from the backing when the locking mechanism lockingly engages the backing.

2. An apparatus according to claim 1 wherein the locking mechanism comprises a lock that, in the locking state of the releasable locking mechanism, permits motion of at least a portion of the backing in the insertion direction from a first side of the lock to a second side of the lock and wherein interaction between the lock and the backing prevents motion of the at least a portion of the backing in the reverse direction from the second side of the lock to the first side of the lock.

3. An apparatus according to claim 1 wherein the locking mechanism is spaced apart from the backing when the forcer is in the retracted state and, wherein, the locking mechanism lockingly engages the backing as the backing travels in the insertion direction in response to the forcer operating from the retracted state to the extended state.

4. An apparatus according to claim 1 wherein the backing comprises at least one concavity and the locking mechanism comprises at least one pawl which extends into the at least one concavity to lockingly engage the backing as the backing travels in the insertion direction in response to the forcer operating from the retracted state to the extended state.

5. An apparatus according to claim 4 wherein the backing comprises a plurality of transversely extending teeth, each transversely extending tooth comprising an insertion-direction face and a reverse-direction face and wherein each concavity is defined by the insertion-direction face and the reverse-direction face of a pair of adjacent teeth.

6. An apparatus according to claim 5 wherein the insertion-direction face of each tooth is shaped such that contact between the pawl and the insertion-direction face of each tooth causes deformation of at least part of the pawl as the backing travels in the insertion direction in response to the forcer operating from the retracted state to the extended state.

7. An apparatus according to claim 5 herein wherein the insertion-direction face of each tooth is shaped such that contact between the pawl and the insertion-direction face of each tooth causes movement (e.g. translation and/or rotation) of at least part of the pawl as the backing travels in the insertion direction in response to the forcer operating from the retracted state to the extended state.

8. An apparatus according to claim 5 wherein the insertion-direction face of each tooth is shaped such that contact between the pawl and the insertion-direction face of each tooth causes deformation of a bias mechanism that biases the pawl toward the backing as the backing travels in the insertion direction in response to the forcer operating from the retracted state to the extended state.

9. An apparatus according to claim 5 wherein the reverse-direction face of each tooth is shaped to guide the pawl into a corresponding concavity and thereby prevent reverse direction movement of the backing.

10. An apparatus according to claim 4 wherein the pawl is shaped to extend transversely and in the insertion direction when the locking mechanism lockingly engages the backing.

11. An apparatus according to claim 4 wherein at least a portion of the pawl is biased in a transverse direction toward the backing when the locking mechanism lockingly engages the backing.

12. An apparatus according to claim 11 wherein the pawl is pivotally mounted and wherein the portion of the pawl is pivotally biased toward the backing when the locking mechanism lockingly engages the backing.

13. An apparatus according to claim 4 wherein at least a portion of the pawl is deformed in a transverse direction away from the backing when the locking mechanism lockingly engages the backing.

14. An apparatus according to claim 4 comprising a pawl release which is actuatable to withdraw the pawl from engagement with the concavity.

15. An apparatus according to claim 14 wherein the pawl release is actuatable to withdraw the pawl from engagement with the concavity by pivotal motion of the pawl.

16. An apparatus according to claim 1 wherein the locking mechanism is releasable by applying force to the housing in a transverse direction nonparallel to the insertion and reverse directions.

17. An apparatus according claim 1 wherein the locking mechanism is releasable by applying force to the housing in the insertion direction.

18. An apparatus according to claim 17 wherein the housing comprises a first section having a first beveled surface and a second section having a second beveled surface and application of force to the housing in the insertion direction causes the first beveled surface of the first section of the housing to contact the second beveled surface of the second section of the housing thereby deforming and/or otherwise moving at least a portion of the second section of the housing to release the locking mechanism.

19. An apparatus according to claim 18 wherein deforming and/or otherwise moving at least a portion of the second section of the housing to release the locking mechanism comprises deforming and/or otherwise moving the at least a portion of the second section of the housing in a transverse direction nonparallel to the insertion and reverse directions.

20. An apparatus according to claim 18 wherein deforming and/or otherwise moving at least a portion of the second section of the housing to release the locking mechanism release comprises deforming and/or otherwise moving the at least a portion of the second section of the housing until one or more stops projecting from the second section of the housing disengage the backing.

21.-76. (canceled)
